(19) United States
(12) Patent Application Publication
(10) Pub. No.: US 2003/0219816 A1
(43) Pub. Date: Nov. 27, 2003

(54) Composite microarray slides

(76) Inventors: Keith Solomon, Northford, CT (US); Mark Meyering, Middlefield, CT (US); Derek Daigle, New Haven, CT (US); Aaron Spearin, Willamantic, CT (US)

Correspondence Address: CUMMINGS & LOCKWOOD, Granite Square, 700 State Street, P.O. Box-1960, New Haven, CT, 06509-1960, US

(21) Appl. No.: 10/410,709

(22) Filed: Apr. 10, 2003

Related U.S. Application Data
(63) Continuation-in-part of application No. 09/898,102, filed on Jul. 3, 2001.
(63) Continuation-in-part of application No. 09/897,333, filed on Jul. 2, 2001.

(51) Int. Cl. C12Q 1/68; B05D 3/00; G01N 33/53; C12M 1/34

(52) U.S. Cl. 435/6; 435/7.1; 435/287.2; 427/2.11

(57) ABSTRACT

Improved composite microarray slides for use in micro-analytical diagnostic applications are disclosed. Specifically, composite microarray slides useful for carrying a microarray of biological polymers on the surface thereof including composite microarray slides having a porous membrane formed by a phase inversion process effectively attached by covalent bonding through chemical agents that comprise anchor/linker moieties to a substrate that prepares the substrate to sufficiently bond to the porous membrane formed by a phase inversion process such that the combination produced thereby is useful in microarray applications and wherein the composite microarray slides are covalently bonded to a solid base member, such as, for example, a glass or Mylar microscope slide, such that the combination produced thereby is useful in microarray applications. Apparatus and methods for fabricating the composite microarray slides are also disclosed.

### RELATED APPLICATIONS

This application is a continuation-in-part of commonly owned U.S. Provisional Patent Application Serial No. 60/216,390 of Amin et al., filed Jul. 9, 2000, entitled “Improved Combination of Microporous Membrane and Solid Support for Micro-Analytical Diagnostic Applications,” U.S. patent application Ser. No. 09/898,102 of Amin et al., filed on Jul. 3, 2001, entitled “Combination Of Microporous Membrane And Solid Support For Micro-Analytical Diagnostic Applications,” U.S. Provisional Patent Application Serial No. 60/216,229 of Andreoli et al., filed Jul. 5, 2000, entitled “IMPROVED NON-LUMINESCENT SUBSTRATE,” U.S. patent application Ser. No. 09/897,333 of Andreoli et al., filed on Jul. 2, 2001, entitled “NON-LUMINESCENT SUBSTRATE,” U.S. Provisional Patent Application Serial No. 60/224,141, entitled “Improved Low Fluorescence Nylon/Glass Composites for Micro-Analytical Diagnostic Applications” of Ostreicher et al., filed Aug. 10, 2000, and U.S. patent application Ser. No. 09/898,102 of Amin et al., entitled “Improved Low Fluorescence Nylon/Glass Composites for Micro-Analytical Diagnostic Applications,” filed on Jul. 5, 2001, the disclosure of each is herein incorporated by reference to the extent not inconsistent with the present disclosure.

### BACKGROUND OF THE DISCLOSURE

The present disclosure relates to improved composite microarray slides useful for carrying a microarray of biological polymers on the surface thereof and, more particularly, to improved composite microarray slides having a porous membrane formed by a phase inversion process effectively attached by bonding through chemical agents that form a surface treatment on a substrate that prepares the substrate to sufficiently bond to the microporous membrane resulting in an attachment layer such that the combination produced thereby is useful in microarray applications and, most particularly, to improved composite microarray slides wherein a porous nylon membrane is covalently bonded to a solid base member, such as, for example, a glass or Mylar microscope slide, such that the combination produced thereby is useful in microarray applications and to a process for producing such improved composite microarray slides.

Varieties of methods are currently available for making arrays of biological macromolecules and biological polymers such as nucleic acid molecules, proteins, or enzymes. One method for making ordered arrays of DNA on a porous membrane is a “dot blot” approach. In this method, a vacuum manifold transfers a plurality, e.g., 96, aqueous samples of DNA from 3 millimeter diameter wells to a porous membrane. A common variant of this procedure is a “slot-blot” method in which the wells have highly elongated oval shapes.

The DNA is immobilized on the porous membrane by baking the membrane or exposing it to UV radiation. This is a manual procedure practical for making one array at a time and usually limited to 96 samples per array. “Dot-blot” procedures are therefore inadequate for applications in which many thousand samples must be determined.

A more efficient technique employed for making ordered arrays of genomic fragments (e.g., PCR products) uses an array of pins dipped into the wells, e.g., the 96 wells of a microtitre plate, for transferring an array of samples to a substrate, such as a porous membrane. One array includes pins that are designed to spot a membrane in a staggered fashion, for creating an array of 9,216 spots in a 22×22 cm area (Lehrach, et al., 1990). A limitation with this approach is that the volume of DNA spotted in each pixel of each array is highly variable. In addition, the number of arrays that can be made with each dipping is usually quite small.

An alternate method of creating ordered arrays of nucleic acid sequences is described by Pirrung, et al. (1992), and by Fodor, et al. (1991). The method involves synthesizing different nucleic acid sequences at different discrete regions of a support. This method employs elaborate synthetic schemes, and is generally limited to relatively short nucleic acid sample, e.g., less than 20 bases. A related method has been described by Southern, et al. (1992).

Khrapko, et al. (1991) describes a method of making an oligonucleotide matrix by spotting DNA onto a thin layer of polyacrylamide. The spotting is done manually with a micropipette.

Roda, et al. (2000) describe a method for producing bidimensional arrays of horseradish peroxidase (HRP) on cellulose paper using a commercial ink-jet printer at a density of 10-100 spots/cm².

None of the methods or devices described in the above prior art are designed for mass fabrication of microarrays characterized by (i) a large number of micro-sized assay regions separated by a distance of 50-200 microns or less, and (ii) a well-defined amount, typically in the picomole range, of analyte associated with each region of the array.

Furthermore, current technology is directed to performing such assays one at a time for a single array of DNA molecules. For example, the most common method for performing DNA hybridizations to arrays spotted onto porous membrane involves sealing the membrane in a plastic bag (Maniatis, et al., 1989) or a rotating glass cylinder (Robbins Scientific) with the labeled hybridization probe inside the sealed chamber. For arrays made on non-porous surfaces, such as a microscope slide, each array is incubated with the labeled hybridization probe sealed under a coverslip. These techniques require a separate sealed chamber for each array, which makes the screening and handling of many such arrays inconvenient and time intensive.

Abouzied, et al. (1994) describes a method of printing horizontal lines of antibodies on a nitrocellulose membrane and separating regions of the membrane with vertical stripes of a hydrophobic material. Each vertical stripe is then reacted with a different antigen and the reaction between the immobilized antibody and an antigen is detected using a standard ELISA calorimetric technique. Abouzied's technique makes it possible to screen many one-dimensional arrays simultaneously on a single sheet of nitrocellulose. Abouzied makes the nitrocellulose somewhat hydrophobic using a line drawn with PAP Pen (Research Products International). However, Abouzied does not describe a technology that is capable of completely sealing the pores of the nitrocellulose. The pores of the nitrocellulose are still physically open and so the assay reagents can leak through the hydrophobic barrier during extended high temperature incubations or in the presence of detergents, which makes the Abouzied technique unacceptable for DNA hybridization assays.

Porous membranes with printed patterns of hydrophilic/hydrophobic regions exist for applications such as ordered arrays of bacteria colonies. QA Life Sciences (San Diego, Calif.) makes such a membrane with a grid pattern printed on it. However, this membrane has the same disadvantage as the Abouzied technique since reagents can still flow between the gridded arrays making them unusable for separate DNA hybridization assays.

Pall Corporation makes a 96-well plate with a porous filter heat sealed to the bottom of the plate. These plates are capable of containing different reagents in each well without cross-contamination. Each well is intended to hold only one target element. Furthermore, the 96 well plates are at least 1 cm thick and prevent the use of the device for many calorimetric, fluorescent and radioactive detection formats which require that the membrane lie flat against the detection surface.

More recently, Pall has launched what it refers to as “Vivid” slides. These slides use a membrane laminated with tape to attach nylon to glass. Alternative platforms use a glass slide that is treated with an organosilane to produce a hydrophobic surface, suitable for microarraying. Examples of alternate glass platforms include GAPS Slides (Corning), Nexterion Slides (Schott Inc), and ArrayIt Slides (Telechem International).

Hyseq Corporation has described a method of making an “array of arrays” on a non-porous solid support for use with their sequencing by hybridization technique. The method described by Hyseq involves modifying the chemistry of the solid support material to form a hydrophobic grid pattern where each subdivided region contains a microarray of biomolecules. Hyseq's flat hydrophobic pattern does not make use of physical blocking as an additional means of preventing cross-contamination.

Several patents have described the use of microarray slides in microarray applications. These include U.S. Pat. No. 5,919,626 entitled, “Attachment of unmodified nucleic acids to silanized solid phase surfaces”; U.S. Pat. No. 5,667,976 entitled, “Solid supports for nucleic acid hybridization assays” and U.S. Pat. No. 5,760,130 entitled “Aminosilane/carbodiimide coupling of DNA to glass substrate,” the disclosure of each is herein incorporated by reference to the extent not inconsistent with the present disclosure.

Microarray slides are well known in the art. Schleicher & Schuell have attempted to attach nylon membrane to a glass slide using glue or similar adhesive in their commercially available CAST™ slides. However, the layer of glue or adhesive adds an increased amount of additional, variable thickness to the nylon membrane/glass slide combination, and the gluing/adhesive process may require the use of a scrim-reinforced nylon membrane. The increased amount of additional, variable thickness caused by the glue/adhesive and the reinforcing scrim results in undesirable extra overall thickness of the nylon membrane/glass slide combination and is a disadvantage in microarray applications. Further, the scrim makes the surface of the membrane of the nylon membrane/glass slide combination uneven and less than ideal from an aesthetic perspective. Even further, the chemistry of the glue or adhesive used to attach the nylon membrane to the glass slide is not necessarily optimal to effectuate the combination, nor is it necessarily compatible with the biomolecules, analytes, solvents or buffer systems for which the product is intended to receive, as it may interfere or react with the analyte or lose integrity by debonding or dissolving in solvents and buffers.

Similarly, other products known to be currently commercially available include: modified glass that binds nucleic acids or proteins without the use of a membrane; Coming GAPS Slides, such as, for example CMT-GAPS™ coated slides; nitrocellulose porous membrane cast onto glass, available from Schleicher & Schuell as FAST™ Slides; scrim-reinforced nylon glued or adhered to a glass substrate such as Schleicher & Schuell CAST™ Slides; nonreinforced nylon membrane taped to a glass substrate, available from Pall corporation as Vivid™ slides. Detailed descriptions of these commercially available products are readily available from the respective manufacturers and are known in the art.

However, in microarray applications, binding nucleic acids or proteins directly to a glass substrate has certain disadvantages. Specifically, a considerably smaller surface area for binding the nucleic acids or proteins is available than with a comparably sized microporous membrane/glass slide combination. The larger the binding surface area, the better the signal strength of the biomolecules or analytes, thereby allowing for the detection of smaller samples of biomolecules or analytes. Also, the porous membrane portion of the microporous membrane/glass slide combination naturally adsorbs the biomolecules or analytes and holds them in place on the microporous membrane/glass slide combination, whereas without the microporous membrane portion of the slide, the biomolecules or analytes would just sit on top of a glass surface, as there is no adsorption of the biomolecules or analytes. It is also likely that the efficiency of immobilization of biomolecule on the glass is substantially less than 100%, and may be less than 50%, when compared to immobilization of the target on nylon. This is important, in that the subsequent detection steps require as much of the possible analyte, or target biomolecule, to be available for (in a DNA detection example) hybridization with the labeled probe.

Following the immobilization, there are typically several liquid immersion steps including blocking, washing, hybridization buffer exposure, etc. Each step has the potential for removing analyte from the glass surface, and decreasing the potential strength of the signal.

Nylon is generally regarded as having the highest biomolecule binding efficiency when compared to other the commercially available polymers or other treated substrates. Nylon is also regarded as providing the highest accessibility of functional groups of the analyte thus bound to the nylon surfaces.

Nylon membranes, a specific species of microporous membranes formed by a phase inversion process, have some advantages over nitrocellulose membranes in that nylon is naturally hydrophilic. Nylon membranes also have a greater protein and DNA binding capacity than nitrocellulose. This increased binding capacity means better signal strength and lower detection thresholds in assays.

Nylon membrane pore structure is more easily controllable than nitrocellulose membrane pore structure, and is more physically robust than the nitrocellulose membranes. Nitrocellulose is more brittle, has more pore variability and is extremely flammable when compared to a nylon membrane. The physical weakness, variability and flammability of the nitrocellulose membranes combine to make nitrocellulose membranes more expensive to manufacture than nylon membranes.

As discussed above, there are at least three main disadvantages to scrim-reinforced nylon glued, taped, or otherwise non-covalently adhered to a glass substrate. First, the glue, tape, or adhesive layer increases the undesirable variable thickness to the combination scrim-reinforced nylon/glass slide. The arraying robots that blot the nylon membranes have narrow spatial tolerances, and any increased variable thickness represents additional uncertainty about accurate positioning of the combination scrim-reinforced nylon/glass slide relative to the arraying robots. The same increased variable thickness problem may affect the focal plane and accuracy of microscopic detection optics, which are typically used in reading the surface of a microarray slide.

The second disadvantage is that the scrim-reinforced membrane on the combination scrim-reinforced nylon/glass slide has an irregular surface on the micro scale. This is an important aesthetic problem, from the standpoint of the end user, since the spot sizes made on the membrane are on a similar scale.

Thirdly, the glue/adhesive and the analyte may not be compatible. Specifically, the adhesive which contains an excess of functionalized moieties for attachment can indiscriminately bind the analyte in a way which makes it unavailable for detection; either by binding to the molecule preventing (in the DNA example) hybridization, or by reversibly binding to the analyte such that the attachment is not permanent, and the analyte is sloughed off in the liquid immersion steps prior to detection. Finally, the adhesive itself can be degraded in the multi-step processes leading to detection, and become, by extraction or other means, a mobile species. The adhesive fragment, if bound to the analyte, may be displaced to a location or area beyond the location of detection, or itself become part of a false background signal, depending on the type of detection operation being performed.

In these types of microarray slides, it is desirable to have a nylon microporous layer that is flat, uniform and thin. In the case of charge modified slides, the degree of charge modification must be uniform over the entire slide surface. In the environment of use, as envisioned for the innovative slides described in the present disclosure, the bond between the nylon and the base member, such as, for example, a glass slide or Mylar sheet, must withstand water, sodium hydroxide, sodium dodecyl sulfate, sodium salt/sodium citrate (SSC), high temperatures and other harsh chemicals and conditions for prolonged periods of time. Because of the high air pressure generated between the nylon membrane layer and the glass substrate when the nylon membrane is wetted, the bond therebetween must also be physically strong.

Currently, functionalized glass microscope slides are the support of choice for microarrays. Limitations of these articles include surface fragility, nonuniformity, low surface capacity for analyte, and limitations concerning spot size, density, and quantitative analysis. Most glass slides provide go and no-go information for presence or absence of a hybridization event, mostly due to low capacity of a flat glass surface. The prior art glass microscope slides described above appear to be incapable of providing for stronger binding, higher capacity and smaller spot footprint for the oligonucleotide probe that is spotted on the microarray surface, as the probe must then be accessible to hybridization events with the target sample of purified and labeled gene fragment or cDNA.

An improved article which is a bonded composite of a flat and smooth glass or plastic substrate (an example is a glass microscope slide) to nylon microporous membrane has already been described in the commonly owned related patent applications mentioned above in the present disclosure. In these commonly owned patent applications, the primary mode of attachment is covalent chemical bonding, a secondary mode being physical surface interlocking, which is assisted by chemical resin curing. In one embodiment, as described in these commonly owned patent applications, surface moieties are provided on the substrate by means of a chemical treatment of the (glass or plastic) substrate surface, using chemical agents as an “anchor,” such as, for example, an aminosilane. The nylon membrane provides its own functional surface, as the terminal functional groups of nylon (amine or carboxyl) are available for bonding as well. Between the treated substrate surface and the terminal functional groups on nylon, a “linker” moiety, such as, for example, a bifunctional epoxy polymer, is introduced. Potential limitations of the described commonly owned patent applications include survival of the composite bond under harsh chemical environments, and the multi-step chemical process required to produce such functionalized glass microscope slides.

Thus, there is a continuing need for relatively flat, uniform and thin, composite microarray slides useful for Micro-Analytical Diagnostic Applications. Such composite microarray slides structure should be naturally hydrophilic. Such composite microarray slides should have properties that are easily controlled. Such composite microarray slides should be more physically robust than the nitrocellulose membrane slides of the prior art. Such composite microarray slides should be relatively easily and economically manufactured. Such composite microarray slides should at least minimize any attachment layer between the membrane and the solid substrate that adds undesirable thickness to the membrane/substrate combination. Such composite microarray slides should include chemical agents that comprise anchor/linker moieties resulting in an attachment layer that has minimal thickness or mass which could add nonuniformity to the overall thickness of the composite microarray slides having a porous membrane formed by a phase inversion process useful in microarray applications. Such composite microarray slides should include chemical agents that comprise anchor/linker moieties resulting in an attachment layer that at least minimizes, if not eliminates, the participation of the attachment layer in the binding or detection of the biological polymer (i.e., analytes including but not limited to nucleic acids or proteins) by a composite microarray slide having a porous membrane formed by a phase inversion process useful in microarray applications. Such composite microarray slides could include a porous membrane formed by a phase inversion process useful in microarray applications which includes chemical agents that comprise anchor/linker moieties resulting in the formation of an attachment layer for connecting the porous membrane to the solid substrate that minimizes the interference of the chemical agents that comprise the anchor/linker moieties used to connect the solid substrate portion to the porous membrane portion used for the detection of analytes. Such composite microarray slides should include a porous membrane formed by a phase inversion process useful in microarray applications which includes chemical agents that comprise anchor/linker moieties resulting in an attachment layer that at least sufficiently reduces, if not eliminates, unacceptable nonuniformity of the overall thickness of the substrate/membrane combination structure. Such composite microarray slides should have a sufficiently regular surface on the micro scale. Such composite microarray slides should provide stronger binding, higher capacity and smaller spot footprint for the oligonucleotide probe that is spotted on the microarray surface than a standard treated glass slide. Such composite microarray slides should eliminate compatibility issues between the chemical agents that comprise anchor/linker moieties resulting in an attachment layer and the analyte.

### SUMMARY OF THE DISCLOSURE

The improved composite microarray slides for microarray analysis of the present disclosure include a porous media having a relatively uniformly smooth surface for analytical and diagnostic applications, which is substantially bonded to a substrate or base member, using chemical agents that comprise surface treatments comprising improved anchor/linker moieties resulting in a flat, uniform and relatively thin, attachment layer being formed between the substrate and the porous media. The porous media, such as, for example, a microporous membrane, has characteristics useful for micro-analytical assays such as microarray platforms used in molecular biological assays of gene arrays. The substrate provides rigidity and strength while the improved chemical agents that comprise the anchor/linker moieties resulting in the attachment layer provide a strong, chemically resistant, substantially permanent (relative to the assay and use) physical attachment of the porous media to the substrate.

An object of the present disclosure is to provide composite microarray slides having a porous membrane formed by a phase inversion process and chemical agents that comprise anchor/linker moieties resulting in an attachment layer that operatively bond the porous membrane to a solid substrate such that the combination produced thereby is useful in microarray applications.

Another object of the present disclosure is to provide composite microarray slides having a porous membrane formed by a phase inversion process which include chemical agents that comprise anchor/linker moieties resulting in an attachment layer that has a minimal finite thickness or mass which provides uniformity to the overall thickness of the composite microarray slides such that the combination produced thereby is useful in microarray applications.

A further object of the present disclosure is to provide composite microarray slides having a porous membrane formed by a phase inversion process which include chemical agents that comprise anchor/linker moieties resulting in an attachment layer that minimizes the chemical agents' interference in the binding or detection of nucleic acid or protein analytes.

Yet a further object of the present disclosure is to provide composite microarray slides having a porous membrane formed by a phase inversion process useful in microarray applications which include chemical agents that comprise anchor/linker moieties resulting in an attachment layer that minimizes the interference of the chemical agents used to operatively bond the solid substrate portion to the porous membrane portion thereof with the detection of analytes such that the combination produced thereby is useful in microarray applications.

Yet another object of the present disclosure is to provide a method for fabricating composite microarray slides having a porous membrane formed by a phase inversion process and chemical agents that comprise anchor/linker moieties resulting in an attachment layer for sufficiently bonding the substrate to a microporous membrane such that the combination produced thereby is useful in microarray applications.

A further object of the present disclosure is to provide composite microarray slides having a porous membrane formed by a phase inversion process which include pigments, such as, for example, carbon-black wherein, such pigmented membranes should reduce the fluorescence of composite microarray slides such that the combination produced thereby is useful in microarray applications.

Still another object of the present disclosure is to provide composite microarray slides having a porous membrane formed by a phase inversion process which include pigments, such as, for example, carbon-black wherein, such pigmented membranes should reduce the reflectance of composite microarray slides such that the combination produced thereby is useful in microarray applications.

Another object of the present disclosure is to provide composite microarray slides having a porous membrane formed by a phase inversion process useful in microarray applications which includes chemical agents that comprise anchor/linker moieties resulting in an attachment layer that significantly reduces, if not eliminates, nonuniformity of the overall thickness of the substrate/membrane combination structure which is associated with using a third component having a finite thickness or mass as the connecting agent such that the combination produced thereby is useful in microarray applications.

Other advantages of the composite microarray slides of the present disclosure, include, but are not limited to, the absence of a reinforcement layer, which has been found to add substantial nonuniformity in flatness and aesthetic properties; the entire surface of the glass can be covered by the membrane, not just a “membrane coupon” (a membrane coupon is a membrane that covers only a fraction of the glass); and the chemistry related to the surface treatment resulting in the attachment layer being formed between the membrane and the substrate may be used successfully with new membrane types, such as, for example, carbon black-filled or Xtra bind™ nylon as disclosed in U.S. patent application Ser. No. 09/873,67, filed Jun. 4, 2001, for NUCLEIC ACID BINDING MATRIX, the disclosure of which is hereby incorporated by reference to the extent not inconsistent with the present disclosure.

In accordance with these and further objects, one aspect of the present disclosure includes composite microarray slides useful for carrying a microarray of biological polymers comprising: a microporous membrane formed by a phase inversion process; a non-porous substrate; and an attachment layer, the attachment layer comprising at least one anchor and at least one linker, the attachment layer being operatively positioned between the microporous membrane and the non-porous substrate, the attachment layer sufficiently bonding the non-porous substrate to the microporous membrane such that the combination composite microarray slide is useful in microarray applications.

Another aspect of the present disclosure includes a method of fabricating composite microarray slides useful for carrying a microarray of biological polymers comprising the acts of: providing a non-porous substrate; providing a microporous membrane formed by a phase inversion process; providing a surface treatment, wherein the surface treatment comprises organosilanes; applying the surface treatment to the non-porous substrate; and

operatively associating the non-porous substrate having the surface treatment applied thereto with the microporous membrane for forming an attachment layer therebetween such that the non-porous substrate is sufficiently bonded to the microporous membrane to withstand challenging environments encountered in microarray applications.

Yet another aspect of the present disclosure may include a post-treatment of the microporous membrane such that the membrane contains a greater positive charge; such a treatment is useful in augmenting the microporous membrane's ability to retain biological polymers, which predominantly are negatively charged.

Other objects and advantages of the disclosure will be apparent from the following description, the accompanying drawings and the appended claims.

### BRIEF DESCRIPTION OF THE DRAWINGS

FIG. 1 is a representative graphic depiction of a representative organosilane, useful with the present disclosure;

FIG. 2 is a representative graphic depiction of the representative aminosilane binding to a glass surface;

FIG. 3 is a representative graphic depiction of the binding of a representative epoxy group with representative carboxyls and amines;

FIG. 4 is a representative graphic depiction of the binding of representative epoxy linkers with a representative aminosilanated slide;

FIG. 5 is a representative graphic depiction of the binding of the representative epoxy linkers to a representative polyamide cross linker;

FIG. 6 is a representative graphic depiction of the binding of a representative nylon membrane to the glass using a representative epoxy linker and a representative polyamide cross linker;

FIG. 7 is a representative graphical depiction of a representative glycidosilane;

FIG. 8 is a representative graphical depiction of a representative polyamide cross linker bound covalently to the glass, through a representative glycidosilane;

FIGS. 9A and 9B are SEMs taken of a representative slide, using the representative epoxy chemistry listed above;

FIG. 10A illustrates a representative generic form of the “anchor” moieties useful with the present disclosure;

FIG. 10B illustrates a representative generic form of a “linker” molecule useful with the present disclosure;

FIG. 10C illustrates a representative generic “curing” molecule, cross linker or secondary linker useful with the present disclosure.

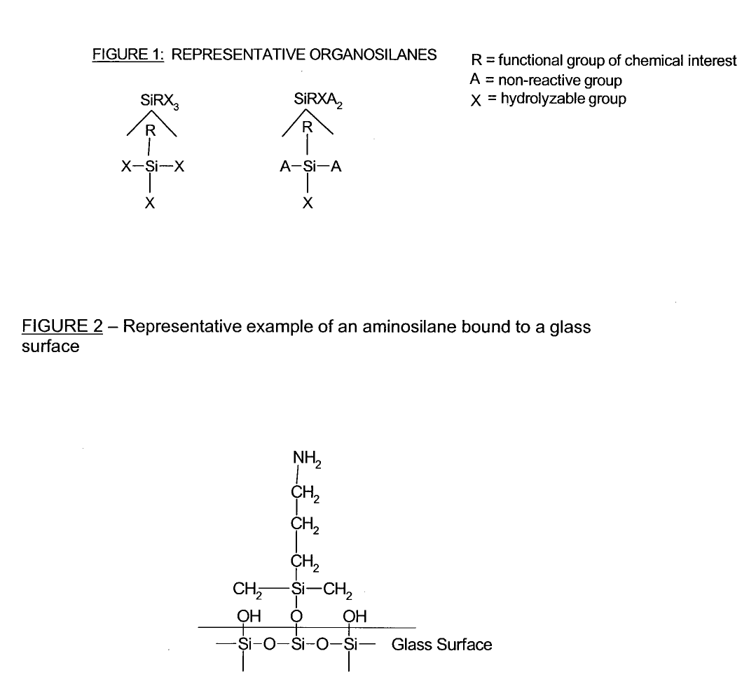

### DETAILED DESCRIPTION OF REPRESENTATIVE EMBODIMENTS

Unless indicated otherwise, the terms defined below have the following meanings:

“Analyte” or “analyte molecule” refers to a molecule, typically a biological macromolecule, such as a polynucleotide (including, but not limited to, DNA, RNA, cDNA, mRNA, PNA, LNA) or polypeptide, or peptide whose presence, amount, and/or identity is to be determined. A biological polymer may be used as an alternate term for a biological macromolecule. The analyte is one member of a ligand/anti-ligand pair. Alternatively, an analyte may be one member of a complimentary hybridization event.

“Analyte-specific assay reagent” refers to a molecule effective to bind specifically to an analyte molecule. The reagent is the opposite member of a ligand/anti-ligand binding pair.

An “array of regions on a solid support” is a linear or two-dimensional array of preferably discrete regions, each having a finite area, formed on the surface of a solid support.

A “microarray” is an array of regions having a density of discrete regions of at least about 100/cm², and preferably at least about 1000/cm². The regions in a microarray have typical dimensions, e.g., diameters, in the range of between about 10-250 m, and are separated from other regions in the array by about the same distance.

A “phase inversion process” is meant to encompass the known art of porous membrane production techniques that involve phase inversion in its various forms, to produce “phase inversion membranes.” By “phase inversion membranes,” it is meant a porous membrane that is formed by the gelation or precipitation of a polymer membrane structure from a “phase inversion dope.” A “phase inversion dope” consists of a continuous phase of dissolved polymer in a good solvent, co-existing with a discrete phase of one or more non-solvent(s) dispersed within the continuous phase. In accordance to generally acknowledged industry practice, the formation of the polymer membrane structure generally includes the steps of casting and quenching a thin layer of the dope under controlled conditions to effect precipitation of the polymer and transition of discrete (non-solvent phase) into a continuous interconnected pore structure. In one manner of explanation, this transition from discrete phase of non-solvent (sometimes referred to as a “pore former”) into a continuum of interconnected pores is generally known as “phase inversion.” Such membranes are well known in the art. Occasionally, such membranes and processes will be called “ternary phase inversion” membranes and processes, with specific reference to the ability to describe the composition of the dope in terms of the three major components; polymer, solvent, and non-solvent(s). The presence of the three major components comprise the “ternary,” system. Variations of this system include: liquid phase inversion, evaporative phase inversion, thermal phase inversion (where dissolution is achieved and sustained at elevated temperature prior to casting and quenching), and others.

The term “silanation” refers to act of grafting or coupling an organosilane via a hydrolyzable functional group on the organosilane to a glass or other surface.

The term “anchor” as used herein describes a molecule comprising an organosilane that contains a hydrolyzable group. The hydrolyzable group is capable of binding to a glass or other surface. There is at least one other group on the organosilane capable of reacting with a terminal group on the linker molecule, including but not limited to, amines, epoxies, glycido, isocyanates, vinyl, and others as would be understood by those skilled in the art.

The term “linker group” or “linker molecule” or “linker” means an organic moiety that serves as a connector between two other molecules. Linkers are typically comprised of a backbone, comprising an aromatic, straight-chain alkyl, or any combination thereof, and a terminal group on either side of the backbone that contains atoms/functional groups such as nitrogen, oxygen or sulfur, a unit such as —NH—, —CH₂—, —C(O)—, —C(O)NH—, or a chain of atoms, such as an alkylidene chain, capable of binding with the compatible terminal group of the anchor, and the other end of the linker capable of binding with the terminal end of the nylon membrane. The terminal ends of the linker that bind respectively to the anchor and to the nylon membrane can be the same or different.

A “chemical agent” is any molecule selected from the group comprising a linear and/or branch chained alkyl, aryl, aralkyl, substituted aryl, a substituted and/or unsubstituted cycloalkyl, and heterocyclic groups, and organosilane.

The term “delamination” or “delaminate” refers to separation of a membrane from a solid substrate.

The term “alkyl” refers to straight or branched chain unsubstituted hydrocarbon groups of 1 to 20 carbon atoms, preferably 1 to 7 carbon atoms. The expression “lower alkyl” refers to unsubstituted alkyl groups of 1 to 4 carbon atoms.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted.

The term “aralkyl” refers to an aryl group bonded directly through an alkyl group, such as benzyl.

The term “substituted aryl” refers to an aryl group substituted by, for example, one to four substituents such as alkyl; substituted alkyl, phenyl, substituted phenyl, heterocyclo, halo, trifluoromethoxy, trifluoromethyl, hydroxy, alkoxy, cycloalkyloxy, heterocyclooxy, alkanoyl, alkanoyloxy, amino, alkylamino, aralkylamino, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy. The substituent may be further substituted by halo, hydroxy, alkyl, alkoxy, aryl, substituted aryl, substituted alkyl or aralkyl.

The term “cycloalkyl” refers to optionally substituted, saturated cyclic hydrocarbon ring systems, preferably containing 1 to 3 rings and 3 to 7 carbons per ring which may be further fused with an unsaturated C.sub.3-C.sub.7 carbocyclic ring. Exemplary groups include cyclopropyl, cyclobutyl, cyclopentyl, cyclohexyl, cycloheptyl, cyclooctyl, cyclodecyl, cyclododecyl, and adamantyl. Exemplary substituents include one or more alkyl groups as described above, or one or more groups described above as alkyl substituents.

The terms “heterocycle,” “heterocyclic” and “heterocycle” refer to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom.

Exemplary substituents include one or more alkyl groups as described above or one or more groups described above as alkyl substituents. Also included are smaller heterocyclos, such as, epoxides and aziridines.

The term “heteroatoms” shall include oxygen, sulfur and nitrogen.

The term “uniform” refers to the regular distribution of the attachment layer on the non-porous substrate, such that after application of the surface treatment, the regularity of the surface of the attachment layer translates into minimal deviations on the upper face of the membrane. Uniform thickness also refers to such regular distribution across the entire length of the non-porous substrate.

Composite microarray slides comprise a porous nylon or other polymer membrane bound to a solid backing, typically a glass microscope slide. Microarray slides are used in gene sequencing and expression analysis applications where thousands of hybridization assays are performed on the surface of a single microarray slide.

When a microporous nylon membrane formed by a phase inversion process is still wet from casting, the nylon membrane has a greater thickness than after being dried. If the membrane is stretched out over a surface and then dried, the nylon membrane shrinks in the direction of thickness. The nylon membrane also binds tightly to the surface it contacts. If the nylon membrane has been dried once and then rewetted, the nylon membrane does not exhibit the binding property described above. More importantly, the nylon membrane loses the binding property once the nylon membrane gets wet after having been tightly bound to a surface.

Given the above characteristic of nylon membrane, it was decided to find mechanisms to attach the nylon membrane to a substrate such that the bond between the nylon membrane and the substrate would remain intact after being exposed to various known severe conditions experienced in actual practice. For example, to be considered for actual commercial applications, nylon/solid composite slides should withstand immersion in 4× sodium salt/sodium citrate (SSC) at 60° C., for at least 15 hours U.S. patent application Ser. No. 09/898,102 of Amin et al., filed on Jul. 3, 2001, entitled “Combination Of Microporous Membrane And Solid Support For Micro-Analytical Diagnostic Applications,” details the disclosure of a system for the binding of a nylon membrane to a substrate, for microarray applications and the resulting composite slide. A typical substrate, as described in this application, was a glass microscope slide, although examples were provided for alternative plastic substrates.

In one representative embodiment of the Amin et al. patent application, surface moieties were provided on the substrate by treatment of the smooth and flat glass substrate surface, using a chemical “anchor” such as, for example, an aminosilane. A linker molecule was introduced such that one end of the linker molecule binds to the anchor molecule, and the other end of the linker molecule binds to the nylon membrane. The nylon membrane provides its own functional surface, as the terminal functional groups (amine or carboxyl) of nylon are available for bonding. Between the treated substrate surface and the terminal functional groups on the nylon, the Resicart E quaternary amine epichlorohydrin polymer was introduced as the linker molecule. The nylon was brought into contact with the linker molecule while the nylon was still wet and swollen. The restrained (in X and Y direction) drying and (Z direction) shrinking of nylon, along with the curing, chemical attachment and bond formation of nylon with the “linker” were believed to be substantially simultaneous. The result was a flat, uniform, and aesthetically acceptable nylon-glass composite, having a minimal bonding layer, and a functional nylon surface presented to the microarray assay. This nylon-glass composite demonstrated good general bond strength, and was used to demonstrate proof-of-principle for innovative chemiluminescent detection systems, under certain conditions.

Unfortunately, a problem was discovered with the bond strength between the nylon and the glass when the slide was immersed in 4×SSC @ 60° C. for more than two (2) hours. After being immersed for more than two (2) hours, the nylon would detach or delaminate from the glass, indicating a breakdown of the bonding layer therebetween. In order to solve or alleviate this problem, a re-optimization of the above chemistries, or additional robust bonding chemistries needed to be developed.

As stated above, the new problem to be solved was the failure of the bond between the microporous membrane and the glass slide after about two (2) hours in 4×SSC @ 60° C. Since all of the other design criteria appeared to have been met using slides provided from the related incorporated by reference patent applications, it was decided to attempt variations on the successful approach in an effort to solve the above problem. In that respect, the concept of using glass/Anchor/Linker moieties was further developed.

The original anchor moieties used in the incorporated by reference patent applications included a triethoxysilane footprint, an n-alkane spacer arm with a terminal amine functionality, such as, for example, 3-aminopropyl triethoxysilane, plus an appropriate carrier solvent system and a simple process for attachment to the glass. In the new and improved composite microarray slides of the present disclosure, the original amine functionality is carried over but with a different reactive moiety attached to the glass for a more uniform surface distribution, such as, for example, 3-aminopropyldimethylethoxysilane. The prior anchor moiety, 3-aminopropyl triethoxysilane, is still functional in the presently preferred representative embodiment.

In contrast with the original linker chemistry, quaternary amine-epoxy wet strength resins (polyamino polyepicholorohydrin resin) such as, for example, Resicart E (Ciba-Geigy), the improved composite slides of the present disclosure include n-glycidyl ethers, such as, for example, 1,4-butanediol diglycidyl ether; aldehydes, such as, for example, glutaraldehyde; acrylics; polyester-silanes; epoxies, such as Bisphenol “A” diglycydl ether; and others, as would be known to those skilled in the art.

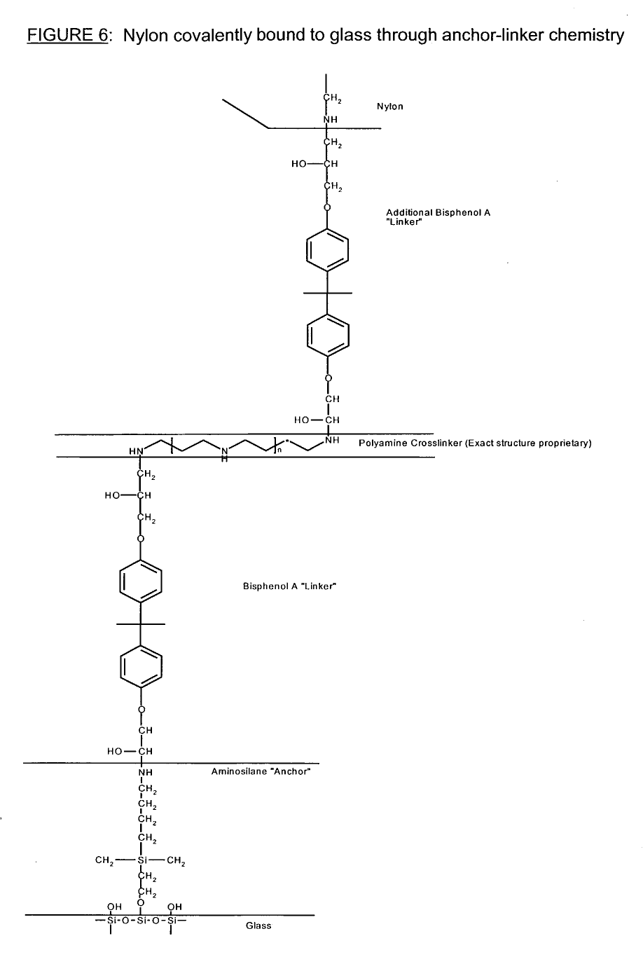

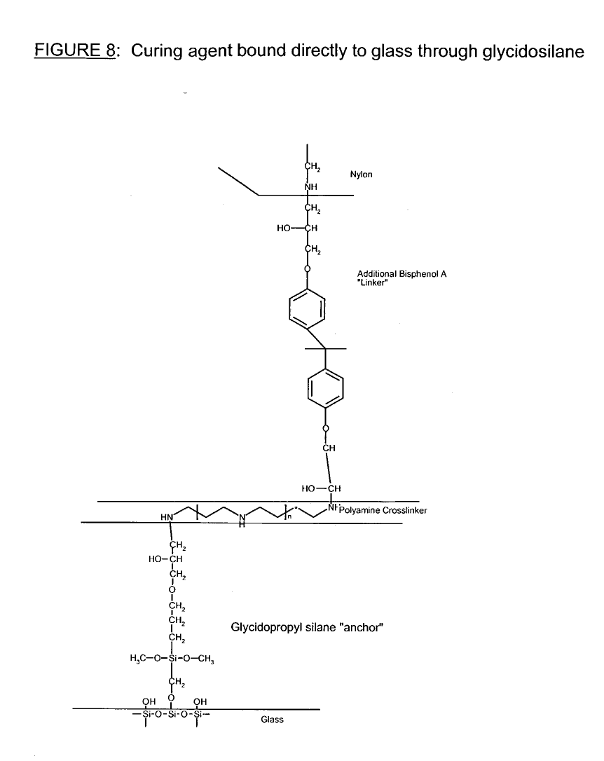

As illustrated in FIGS. 6 and 8 of the Amin et al. application, the organosilane was the chemical agent that was characterized as the “anchor” molecule connected to a linker molecule, which is optionally bonded to a polyamine cross linker which allows binding of additional, optional linker molecules. The terminal linker is likely covalently bonded to nylon. In the case of Adcote 89R3 (Example 3), it is likely that the bonding occurs via hydrogen bonding, though this is unconfirmed. Thus, the bonding to the nylon can be via hydrogen bond, van der Waals bonding, or preferably, covalent bonding.

It is believed that there are several alternative linker chemistries (and combinations of anchor, linker, and optional cross linker moieties) which will deliver robust performance but were not explored in the above mentioned incorporated by reference patent applications. It is believed that, with further experimentation, proper formulations and application conditions, as gleaned from the failure under the conditions described above, can be found to deliver superior bond strength between the porous membrane and the solid substrate such that the composite microarray slides of the present disclosure would withstand immersion in 4× sodium salt/sodium citrate (SSC) at 60° C., for at least 15 hours With respect to the original cross linker chemistry, the original cross linker provided for insoluble, immobile bonding using an appropriate solvent and a polyamine, such as, for example, Tetraethylene Pentamine (TEPA), improved cross linkers utilize a proprietary “hardener” formula, such as, for example, Epikure 3125, Epikure 3115, or Epikure 8535-W50, obtained from Resolution Performance Products, Houston, Tex.

The proposed composite microarray slides of the present disclosure offer equivalent aesthetic properties to the Resicart E system, as described in the aforementioned patent applications, but use an alternate linker for improved bond survivability in SSC and organic solvents such as DMF. The present disclosure addresses the problem in the art of other types of adhesive layers which add variable thickness to the combination scrim-reinforced nylon/glass slide. The attachment layer of the present disclosure, comprising the anchors and linkers described herein, provides a flat, uniform and relatively thin surface in attaching the membrane to the slide. Such uniformity in the attachment layer results in minimal deviations in the upper surface of the membrane.

The following is a general description of such a representative improved modified composite microarray slides of the present disclosure and will be described by way of the general description below:

First, a glass slide is selected, and cleaned, via any suitable means, as would be understood by one skilled in the art. Following cleaning, a chemical agent that performs the anchor function is applied to the glass slide, rinsed to remove any excess material or reagent, and cured, via an ambient cure, elevated temperature cure, or any combination thereof as would be understood by one skilled in the art. One suitable chemical that functions as an anchor is 3-aminopropyl triethoxysilane. After the excess material/reagent has been removed and the remainder is cured on the glass slide, a solution of a suitable chemical reagent that performs the “linker” function is prepared, as follows.

One presently preferred chemical reagent that functions as a linker for utilization with the new and improved system of the present disclosure is a Bisphenol A type epoxy, commercially known as Epon 828.

To effectuate curing, any number of curing agents may be used, but at this point, utilization of a polyamide based curing agent, particularly Epikure 3115, is presently preferred. The two components are mixed, using any suitable means, as would be understood by those skilled in the art. Finally, a suitable epoxy-functional silane may be added to the above described mixture of chemical reagents. One such, presently preferred, epoxy-functional silane is 3-glycidopropyltrimethoxysilane. Once mixed, all three of the above described chemical components are dissolved in a suitable solvent, such as, for example, xylene, for application to the glass slide. A thin layer of the epoxy mixture is then applied to the glass slide via spin coating. The nylon microporous membrane is then operatively positioned relative to the treated glass slide, restrained in the x-and-y directions, and then oven-cured, as would be understood by those skilled in the art.

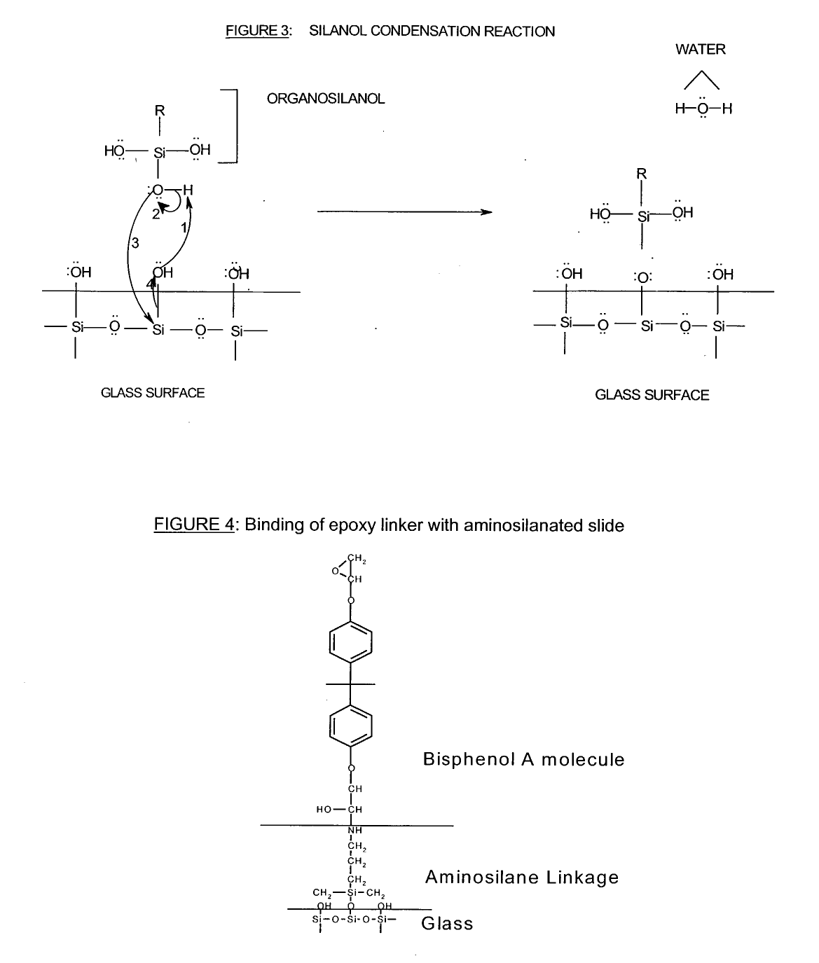

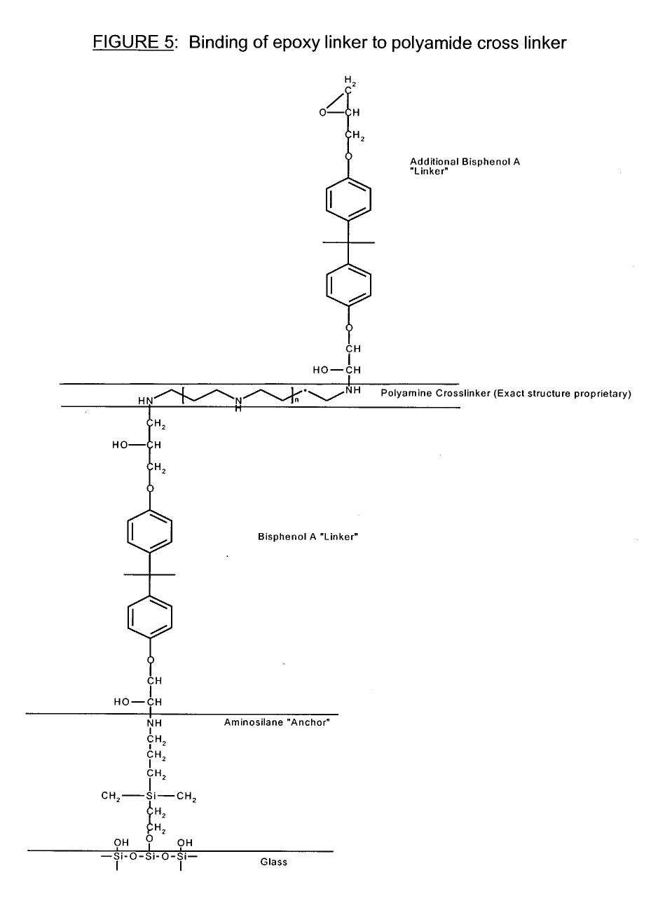

While not wishing to be bound by theory, it is presently believed that the aminosilane will react with the glass, as illustrated in FIG. 3. It is well known to those in the art that nucleophilic amines react with epoxy groups. Hence, the aminosilane of the “anchor” will open the epoxide ring of Bisphenol A to form the coupled product as illustrated in FIG. 4. The opposite end of the Bisphenol A molecule will then react with the free amine on the polyamide curing agent, to form the structure as illustrated in FIG. 5. Once nylon membrane is laid onto the glass, the amine groups on the nylon are believed to react with the terminal epoxy groups on the Bisphenol A, to produce the complete structure as illustrated in FIG. 6.

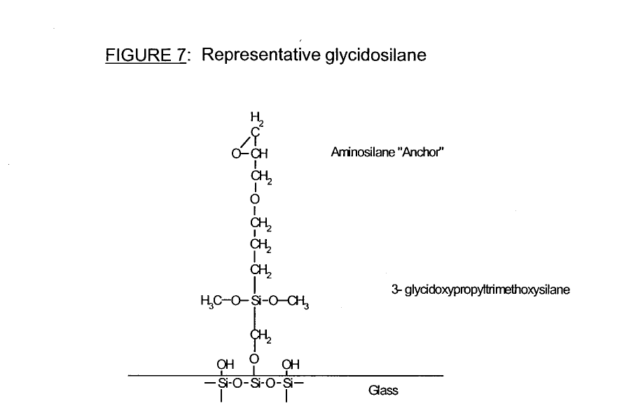

FIG. 7 illustrates a typical glycidosilane. It is believed that the free glycidosilane will react with the curing agent in solution, and then bind to any unsilanated sites remaining on the glass, as illustrated in FIG. 8. Balancing the epoxy, amine, and silane ratios in the mix is delicate and is believed to impact the ultimate bonding strength of the composite attachment layer formed thereby. It should be noted that glycidosilane is presently believed to be an optional component. It is presently believed that sufficient bond strength can be demonstrated in formulations with or without the glycidosilane. One other advantage to using the glycidosilane is the apparent improvement in the working time of the linker chemistry prior to its application to the anchor.

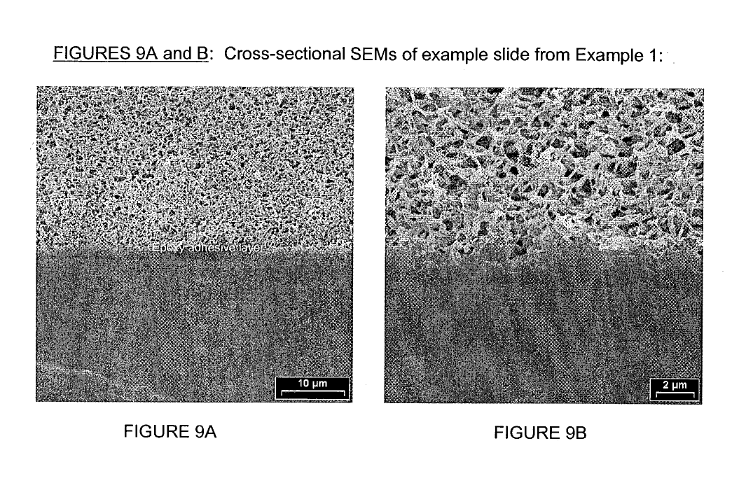

FIGS. 9A and B are scanning-electron micrographs (SEM) of a typical slide produced using the above described chemistry. Note that the thickness of the adhesive layer between the two other components, the glass and the microporous membrane, is only about 1-2 microns, well below the about 10 microns usually required for most commercial adhesives. FIGS. 9A and B demonstrate that the thin, uniform attachment layer adds minimal deviations, if any, to the upper surface of the membrane, which is an advantage over the variable thicknesses offered by other adhesive layers, such as glue.

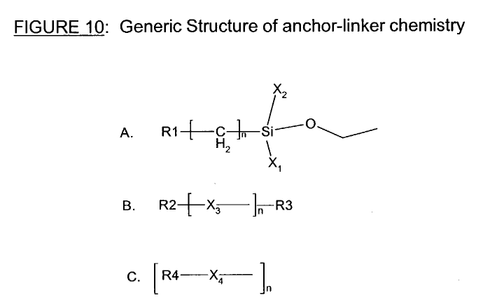

FIG. 10A illustrates a generic form of the “anchor” moieties used in the present disclosure. This particular representation is an organosilane, of any chain length designed to bind via a functional group onto the surface of the glass. The silane may contain one ethoxy group for binding to glass (in which case groups X1 and X2 are usually alkanes, usually 1-2 carbons in length), or have additional ethoxy groups in positions X1 and X2. RI is selected from a series of functional groups that will bind with the “linker” at R2 in the next step. These chemical agents include, but are not limited to, amines, epoxies, glycido, isocyanates, vinyl, and other functional groups as would be a understood by those skilled in the art.

Note that the “anchor” moieties may also be included in the linker moieties by coupling a silane group directly to the linker molecule, as with the Adcote 89R3 chemistry.

FIG. 10B illustrates a generic form of a “linker” molecule. X3 represents the “backbone” of the linker, and may be aromatic, straight chain, or any combination thereof. Generally, linkers are polymers, with saturated polymers generally preferred for improved chemical resistance. R2 is selected such that it binds with the functional group, R1, of the “anchor” in FIG. 10A, and is generally selected from the group including, but not limited to, the following: amines, epoxies, acrylics, isocyanates, glycido, esters, and others.

R3 is selected to bind with the nylon microporous membrane, and may be identical to R2. Generally, covalent binding with the functional groups (amines and carboxy groups) on the nylon is presently preferred; however, this is not required. Alternately, mechanical interlocking of the linker with the nylon membrane is also sometimes sufficient for good bond strength adhesion of the nylon-glass layer.

FIG. 10C illustrates a generic “curing” molecule, cross linker, or secondary linker. The secondary linker can be any molecule containing at least two functional groups that are capable of binding to the linker. The purpose of this molecule is to add additional length to the linker molecule by crosslinking with the linker to create a matrix, enabling the linker to better penetrate into the pore structure of the membrane. This “secondary linker” may be eliminated in some representative embodiments of the present disclosure, such as Example 3 (Adcote 89R3). Unfortunately, secondary linker structures are generally proprietary in nature, thus, the exact chemical compositions are not readily discernible or available to the public. In FIG. 10C, X4 may be either aromatic or aliphatic, or any combination thereof, or any molecule which should contain a repeating functional group that will bind with the target linker molecule. R4 represents a suitable functional group, selected for attachment with either the R2 or R3 group on the primary linker molecule. A cross linker molecule may also serve the function of a secondary linker, in which it would have the capability of binding to the nylon.

Composite microarray slides produced utilizing the above described process have demonstrated superior survivability in SSC hybridization solutions, even in overnight exposures at about 60° C. whereas certain competitive slides (S&S CAST slides) delaminate in these conditions in less than 2 hours. The aesthetic appearance of the above described composite microarray slides are not believed to be adversely affected by the minimal attachment layer between the glass slide and the microporous membrane as a result of the chemical agents that act as anchors and linkers, since the chemical agents that act as anchors and linkers are applied as a very thin coating (approximately ˜1 μm) to the non-porous substrate, presently preferably, glass. Similarly produced glass sides are also unaffected by organic solvents, such as DMF, which are known to dissolve many of the commercial adhesives available for use as connecting mechanisms between the glass slides and the microporous membrane.

In accordance with the present disclosure, there are many possible variations to the disclosed chemical agents that comprise a surface treatment for providing an attachment layer between the porous membrane and the substrate that would be known to those skilled in the art including, but not limited to, modifications to the silane (anchor) moieties. Additionally, either the aminosilane or the glycidosilane may be omitted from the chemical agents that comprise the surface treatment system resulting in the attachment layer of the present disclosure. Further, many alternate functional groups on the silanes may be used for reactivity with glass, including, but not limited to, amines, epoxies, and many others.

Concerning the linker moieties, using the Epon 828 Bisphenol A type chemistry is but one of a plurality of possibilities. Other linkers believed feasible, using the same “anchor-linker” chemical agents, include, but are not limited to, acrylics, polyester-silanes, polyesters, alternate epoxies, isocyanates, and equivalents.

Concerning the method of application of the chemical agents on the surface treatment resulting in the attachment layer, spin-coating is only one of a plurality of possible methods of applying the surface treatment to the surface of the substrate. Other possibilities include, but are not limited to, drawdown (knife-style), spraying, coating with a slot-die, or equivalents. The presently perceived primary advantage of spin-coating is the resulting high uniformity of application of chemical agent comprising the surface treatment on the micro scale.

Concerning the membrane type, high and low amine nylon 6,6 have been successfully tested with the chemical agents that comprise the anchors and linkers resulting in the attachment layer of the present disclosure; however, alternate membrane types, including but not limited to, alternate nylons (such as, for example, nylon 4,6) are considered to be within the scope of the present disclosure. Additionally, the use of alternate polymer types may also be feasible, as would be understood by one skilled in the art, including, but not limited to polysulfone, polyethersulfone, polyvinylidenediflouride (PVDF), and nitrocellulose.

In the practice of the present disclosure, the membrane may be applied either wet or dry. Use of wet membrane is presently preferred for added bond strength and uniformity of attachment between the membrane and the substrate.

In the practice of the present disclosure, the membrane may be charged or uncharged and the pore size and thickness of the membrane can be manipulated to any desired range, as would be understood by one skilled in the art.

### EXAMPLE 1

Method for the Attachment of Nylon Membrane to a Glass Substrate: Bisphenol A

Production of Nylon/Glass Composite slides useful as a composite microarray slides for carrying a microarray of biological polymers was carried out as follows.

This representative Example describes the process for producing a sample batch of the nylon/glass composite slides. The representative nylon/glass composite slides which were produced were comprised of a thin (˜2 mil) layer of porous nylon membrane operatively bound to the surface of a three-inch (3″) by one-inch (1″) glass microscope slide. Such slides have proven operable as composite microarray slides useful for carrying a microarray of biological polymers.

The representative process was initiated by dissolving one packet of NoChromix (Godax Labs, Inc) into about 2.5L of concentrated sulfuric acid, then stirring thoroughly until all crystals were dissolved to produce a cleaning solution. Next, the previously prepared cleaning solution was poured into a glass dish (Thermo Shandon model 102), and allowed to sit for about 10 minutes. Glass microscope slides (Erie Scientific #C16-5218) were placed into a 20 slide rack (Thermo Shandon model 100) and then immersed in the cleaning solution, above, for about 30 minutes, then transferred to another dish filled with about 18 mΩ DI water where they remained for about 20 minutes. The slides were then dipped briefly in HPLC grade denatured ethanol (Brand-Nu #HP612) and then silanated by the procedure described below. Alternately, the slides may be cleaned with an about 1 wt % solution of Alconox in DI water, air agitated for about 30 minutes, followed by about a 30 minute sparge with frequently refreshed baths of 18 mΩ DI water.

The slides were silanated by the following representative procedure: First, an about 100 mL solution of about 95% ethanol and about 5% water (percent by volume) was prepared. Then, about 2 mL of 3-aminopropyldimethylethoxysilane (United Chemical Technologies #A0735) was added to the above solution, mixed thoroughly, and allowed to sit for about 5 minutes. After the preceding about 5 minute activity was complete, the resulting solution was poured into glass dish, and the slides were immersed therein for about 2 minutes. The slides were then removed from the silane solution, dipped into a dish containing ethanol for about 7 seconds, and removed from the dish. The slides were then placed into an oven for about 10 minutes at about 110° C., and allowed to finish reacting overnight.

It was determined during the previous step that excessive rinsing of microslides with ethanol appeared to disrupt the temporary hydrogen bonding of the silane prior to cure, resulting in diminished bonding.

After the reactions were finished, the slides were inspected for visual blemishes or other imperfections. Any of the slides with visual blemishes or other imperfections were rejected and not used.

The next day, a representative Bisphenol A “linker” solution was made by adding the following to a 250 mL Erlenmeyer flask and mixing thoroughly after each step in which a new ingredient was added:

about 10 grams Epon 828 (a Bisphenol A type epoxy resin); and

about 34 grams Xylene.

In a separate 250 mL Erlenmeyer flask, the following were also added:

about 4.1 grams Epikure 3115 (a polyamide based curing agent);

about 34 grams Xylene; and

about 1.8 grams 3-glycidopropyltrimethoxysilane.

The contents of the first flask (epoxy) were then poured into the second flask, sealed, and agitated with a lab stirrer for about an additional about 15 hrs at about 60° C. The resultant solution from the combination of the two flasks described above resulted in an about 12 wt % Bisphenol A “linker” solution.

During this step, it was determined that reaching a minimum time of mixing appeared to be related to producing composite slides with acceptable aesthetic properties and adhesion. It was also determined that optimal results were achieved if the epoxy was stored, in a closed vessel, at about 60° C. for about one to about three days prior to mixing.

It is believed that storing the epoxy in this manner prevents recrystallization of the epoxy solution, which may lead to bumps in the coating used as the surface treatment. This particular representative solution had a shelf life of about 30 hours, beyond which point when applied, the solution became unusable for the intended purpose. Following the mixing cycle, a single cleaned and silanated slide was then placed on a spin coater (Specialty Coating Systems model P6708). Surface was flooded with the epoxy solution prepared above, then allowed to spin at the following cycle:

| RPM | Time (seconds) |
|---|---|
| ˜500 | ˜10 |
| ˜900 | ˜10 |
| ˜3000 | ˜20 |

Next, the slides were removed from the spin coater, and placed on a 5 inch×10 inch metal plate. This spin-coating cycle was then repeated for two (2) additional slides. Next, wet-as-cast porous nylon membrane (as described in U.S. Pat. Nos. 3,876,738 and 4,707,265) was operatively positioned over the slides and stretched. Personnel wearing gloves handled the wet-as-cast porous nylon membrane. The wet-as-cast porous nylon membrane used had been cast, quenched, and washed with DI water, but had not yet been exposed to a drying step, hence the term “wet-as-cast.” The wet-as-cast porous nylon membrane had a thickness of approximately 1.5 mils, a nominal pore size less than about 0.2, and a target initial bubble point in water of about 135 PSI (once dried). The base polymer for this wet-as-cast porous nylon membrane is Vydyne 66Z nylon (Solutia, Inc), which is a high molecular weight nylon that is preferentially terminated by amine end groups.

During the application of the wet-as-cast porous nylon membrane to the treated slides, care was taken to ensure removal of any air bubbles between the wet-as-cast porous nylon membrane and each slide. The wet-as-cast porous nylon membrane was flattened onto each slide and all wrinkles were removed.

Once positioned on the slides, the wet-as-cast porous nylon membrane was clipped into position, as is known in the art. The entire assembly was then heated in a convection oven at about 110° C. for about 45 minutes. After heating, the excess, now dried, porous nylon membrane was removed from the slides by trimming, as is known in the art.

Following trimming, the slides were allowed to sit overnight, in order for the epoxy resin to further cure. To test the adhesive strength of the membrane to the substrate by the attachment layer produced utilizing the above process, a solution of 4× SSC (sodium salt, sodium citrate) was prepared by diluting a stock 20× solution (Sigma # S6639).

The slides were placed into a Tupperware container, SSC solution was poured on top of the slides, and the container was sealed. The container was then placed in a hybridization oven at about 60° C. for a minimum of about 12 hours with gentle rocking.

Upon removal from the solution, all the membrane components of the composite slides were found to be securely bonded to the substrate component, with no delamination of the membrane from the substrate. The slides that were exposed for a longer period at 60° C., in excess of 72 hours, also showed no delamination of the nylon from the substrate.

Further testing of adhesion between the membrane and the substrate was accomplished by the following method: first, two (2) slides were selected and placed in a 60 mL vial. Next, a solution of n-dimethylformamide (DMF, Aldrich 31,993-7) was poured over slides, and the lid sealed. DMF is an aggressive solvent that can be used to apply a variety of chemistries to the surface of slides, and is known to attack common adhesives such as acrylates, urethanes, and polyesters. The slides were allowed to sit at room temperature for a minimum of about 6 hours, then removed and rubbed firmly.

After the above treatment, the slides exhibited no loss of adhesive strength of the bond between the membrane and the substrate after immersion in DMF, even after exposure at room temperature for about 2 weeks.

### EXAMPLE 2

Method for the Attachment of Nylon Membrane to a Glass Substrate: Bisphenol A/Epikure 3125

Production of Nylon/Glass Composite slides useful as a composite microarray slides for carrying a microarray of biological polymers was carried out in the same manner as Example 1, with the following exceptions:

Formulation of a representative epoxy solution was as follows:

about 10 grams Epon 828 (a Bisphenol A type epoxy resin); and

about 35 grams Xylene.

In a second 250 mL Erlenmeyer flask, the following were also added:

about 6 grams Epikure 3125 (a polyamide based curing agent);

about 35 grams Xylene; and

about 1.8 grams 3-glycidopropyltrimethoxysilane.

The representative Epoxy solution was poured into the second flask, and the solution mixed for about five (5) hrs at about 60° C. The solution was then mixed and applied to slides in a similar manner as Example 1. One notable difference from the representative solution of Example 1 is that the resulting solution of Example 2 was ready for use in a shorter time, but had a working life of only about three (3) hours.

The representative slides made according to the procedure described above also survived an overnight immersion in 4×SSC, at about 60° C.

### EXAMPLE 3

Method for the Attachment of Nylon Membrane to a Glass Substrate: Adcote 89R3 (Obtained from Rohm and Haas)

This representative Example describes another representative process for producing a sample batch of nylon/glass composite slides. The nylon/glass composite slides which were produced were comprised of a thin (˜4 mil) layer of porous nylon membrane operatively bound to the surface of a three-inch (3″) by one-inch (1″) glass microscope slide. Such slides have proven operable as a composite microarray slides useful for carrying a microarray of biological polymers.

Production of Nylon/Glass Composite slides useful as a composite microarray slides for carrying a microarray of biological polymers was carried out as follows:

The representative process was initiated by dissolving one packet of NoChromix (Godax Labs, Inc) into about 2.5L of concentrated sulfuric acid, then stirring thoroughly until all crystals were dissolved. Next, the resulting solution was poured into a glass dish (Thermo Shandon model 102), and allowed to sit for about 10 minutes. Glass microscope slides (Erie Scientific #CI6-5218) were placed into a 20 slide rack (Then-no Shandon model 100). The slides were immersed in the acid-NoChromix cleaning solution, above, for about 30 minutes, then transferred to another dish filled with about 18 mΩ DI water for about 20 minutes. The slides were then dipped briefly in HPLC grade denatured ethanol (Brand-Nu #HP612), then removed and placed in an oven for about 10 minutes at about 110° C.

Next, the slides were silanated by the following procedure: First, an about 100 mL solution of about 95% ethanol and about 5% water (percent by volume) was prepared. Next, about 2 mL of 3-aminopropyldimethylethoxysilane (UCT # A0735) was added to the above solution, mixed thoroughly, and allowed to sit for about 5 minutes. Next, the resulting solution was poured into glass dish, and the slides immersed for about 2 minutes. The slides were then removed from silane solution, dipped into a dish containing ethanol, and removed from the dish. This was repeated for a second dish of ethanol, for a total immersion time of about 7 seconds. After removal from the ethanol dish, the slides were placed into an oven for about 10 minutes at about 110° C., then allowed to finish reacting overnight.

During this step, it was found that excessive rinsing of microslides with ethanol appeared to disrupt temporary hydrogen bonding of the silane prior to cure, resulting in diminished bonding.

After a minimum of about four hours, the four slides were inspected for visual blemishes or other imperfections. Any of the four slides with visual blemishes or other imperfections were rejected and not used.

The next day, about a 20 wt % solution of Adcote 89R3 solution was made by adding the following to a 250 mL beaker and mixing thoroughly after each step in which a new ingredient was added:

about 10 grams Adcote 89R3 (Rohm and Haas); and

about 40 grams Toluene (Brand-Nu #9460-03).

At the conclusion of the above step, the silanated slides previously mentioned were then measured using a snap gauge (Mitutoyo model 7326), and then grouped by thickness in 0.2 mil increments. The slides were then placed, in groups of 5, onto a glass plate. A knife-edge style drawdown device (Paul Gardner model #A-P-MO6) was then placed over slides and adjusted to the minimum gap necessary to clear all five (5) slides. Once the minimum gap was determined, the gap was increased by about one mil, to achieve a suitable layer of liquid on the surface of the slide. After making the gap adjustment, about 3 mL of linker solution was then dropped onto the first slide. Next, the liquid was “drawn-down” over all five (5) slides at a rate of about 10 inches per second. This delivers a thin coating of Adcote onto the surface of the slides. After drawing the solution onto the slides, the slides were then immediately placed on a metal mesh plate.

It was determined that during this step the properties of gap clearance and the percent solids of the adhesive mix were all related to achieving acceptable aesthetic properties of the finished slide. In addition, these conditions also affected the strength of the bond between the membrane and the slides.

Next, wet-as-cast porous nylon membrane (as described in U.S. Pat. Nos. 3,876,738 and 4,707,265) was operatively positioned over the slides and stretched. Personnel wearing gloves handled the wet-as-cast porous nylon membrane. The wet-as-cast porous nylon membrane used had been cast, quenched, and washed with DI water, but had not yet been exposed to a drying step, hence the term “wet-as-cast.” The wet-as-cast porous nylon membrane had a nominal pore size of about less than 0.2 microns and a target initial bubble point of about 135 PSI (once dried). The base polymer for this wet-as-cast porous nylon membrane is Vydyne 66Z nylon (Solutia, Inc), which is a high molecular weight nylon that is preferentially terminated by amine end groups.

During the application of the wet-as-cast porous nylon membrane to the treated slides, care was taken to ensure removal of any air bubbles between the wet-as-cast porous nylon membrane and each slide. The wet-as-cast porous nylon membrane was flattened onto each slide and all wrinkles were removed.

Once positioned on the slides, the wet-as-cast porous nylon membrane was clipped into position, as is known in the art. The entire assembly was then heated in a convection oven at about 110° C. for about 45 minutes. After heating, the excess, now dried, porous nylon membrane was removed from the slides by trimming, as is known in the art. Following trimming, the slides were allowed to sit overnight, in order for the polyester linker to further cure.

To test the adhesion of the membrane to the substrate, a solution of 4×SSC was prepared by adding the following to a 500 mL Erlenmeyer flask:

about 40 mL 20×SSC (stock solution, Sigma-Aldrich, S6639) and

about 160 mL DI H₂O.

The slides were then placed into a Tupperware container, solution poured on top of the slides, and the container was sealed.

The container was then placed in a hybridization oven at about 60° C overnight for a minimum of about 12 hours with gentle rocking.

Upon removal from the solution, all membrane was found to be bonded securely to the substrate, with no delamination from the substrate. The slides that were exposed for a longer period at about 60° C., in excess of about 72 hours, also showed no delamination from the substrate.

### EXAMPLE 4

Production of gray microporous membrane composites, having low reflectance and fluorescence, and resistant to hybridization chemistries, was accomplished via the following representative method:

A casting dope was prepared. Methods and systems for preparing the dope used to produce microporous membrane are known in the art. A number of the known prior methods of dope preparation are discussed in representative U.S. Pat. No. 3,876,738 issued Apr. 8, 1975, U.S. Pat. No. 4,340,480 issued Jul. 20, 1982, U.S. Pat. No. 4,770,777 issued Sep. 13, 1988, and U.S. Pat. No. 5,215,662 issued Jun. 1, 1993, the disclosure of each is herein incorporated by reference to the extent not inconsistent with the present disclosure. Modifications of dope making procedures to effectively incorporate a pigment into the casting dope are detailed in commonly owned, co-pending patent application Ser. No. 09/897,333, but scaled to larger volumes as described here: First, about 0.948 lb carbon black (Degussa-Huls product Printex U) was dispersed into about 13.9 lb of formic acid, using a Silverson high-shear mixer (model # L4T-SU, with 1 liter SS mix chamber). Dispersion was accomplished by dividing the carbon black and formic acid into individual aliquots of 50 grams of carbon black dispersed in about 700 g of Formic Acid. Eight (8) aliquots were prepared, followed by a single aliquot of 30 g carbon black dispersed in about 700 g formic acid. The separate aliquots of dispersed carbon black were combined in a transfer vessel, for a total of 430 g (approximately 0.948 lb carbon black) in a total of 6320 g (approximately 13.9 lb of formic acid). Next, about 241.0 lb formic acid were added to a sealed, water jacketed, stainless steel turbine mixer style dope vessel of 40 gallon capacity, and mixed at low speed (150 RPM) with about 24.5 lb of methanol nonsolvent, for about 15 minutes.

Following mixing of the formic acid and methanol, the previously dispersed carbon/formic acid mixture was poured into the vessel, and allowed to mix at low speed (about 150 rpm) for about 2 minutes. Next, about 47.3 lb of Vydyne 66Z (Solutia, Inc), a high amine, high molecular weight nylon 6,6, was added to the formic/carbon/methanol mixture, and allowed to mix at about 450 rpm for about 4 hrs at about 28° C. The above comprised the preparation of the representative nylon “dope.”

A small portion (approximately 20 ml) of the representative dope was subsequently cast and quenched in a laboratory apparatus to simulate the casting process described in U.S. Pat. No. 3,876,738, to produce a single layer, non-reinforced microporous nylon membrane, of about 5 mils thickness in the wet-as-cast state. The wet-as-cast membrane was washed in DI water, and was folded over such that both exposed outer surfaces represented the quenching side of the wet-as-cast membrane, and was dried under restraint to form a dry double layer membrane. The dry double layer had a thickness of about four (4) mils. The L (lightness) value of the dry surface of the dry double layer membrane sample was determined using a Macbeth Coloreye 3100 calorimeter, as described in the above mentioned commonly owned, co-pending patent application, and was found to be approximately 50 units (on a scale of 0 to 100, using the D65 bulb).

The pore size was determined by wetting the dry double layer membrane sample in a mixture of 60% Isopropyl Alcohol, 40% water (by volume), and testing for the previously described Foam All Over Point (FAOP). The resulting FAOP was approximately 55 psi, indicating a microporous membrane with nominal membrane pore size smaller than about 0.2 microns, according to industry standards.

Next, the representative dope was cast using a horizontal drum-type caster, using the methods disclosed in U.S. Pat. No. 3,876,738. Membrane thickness was adjusted by varying the gap between the casting knife and the drum, and was gradually reduced from 10 mils wet thickness until a final membrane wet thickness of 5.5 mils was achieved. The wet single layer membrane was doubled-up, and dried under restraint as before. Membranes were found to have a dry foam-all-over-point (FAOP) in 60/40 v/v IPA/H₂O of about 50 psi, and a thickness of about 4.0 mils (about 2 mils single-layer).

The above steps describe a procedure for making a wet, swollen, microporous membrane, having a gray color and carbon black evenly dispersed among the pore structure. The aforementioned microporous membrane was then attached to slides in the same manner as Example 1. The slides were cleaned, silanated, an epoxy layer spread uniformly over surface, and then the 2 mil thick wet nylon membrane was laid over slides and restrained in the x-and-y directions. The slides were then oven dried, trimmed, and then inspected for aesthetic defects, and tested for L-value.

A surprising result of the experiment was the discovery of color difference that related to the surface orientation of the membrane. Samples of the single layer membrane were attached to glass as described above, with the casting drum side of the membrane oriented downward toward the glass, and the quench side surface oriented upward (the exposed surface). In this orientation, the exposed quench surface was measured for color, and found to have an L-value of approximately 48 units.

Samples were prepared in the alternative arrangement (with quench side attached to the glass, and drum side facing up). These samples were found to have an average L-value of about 58 units. Although this effect of orientation is not presently completely understood, it is noted that the flatness and texture of the surfaces of the membrane are affected by their orientation.

The pore structure of nylon microporous membrane made by these techniques is normally symmetric and isotropic with respect to pore structure (i.e. skinless), but it has been noted that the surface flatness is affected by the presence of a casting substrate. A polished stainless steel drum used as a casting substrate will result in a more glossy appearance to the membrane surface quenched in contact with the stainless steel drum. The opposite surface (i.e. the quench fluid facing surface) has a less glossy appearance, indicating a more 3-dimensional surface texture. Thus, a casting dope with uniform distribution of pigment may display an apparent color difference once quenched, which is affected by surface texture. In the present case, the less textured drum side shows a higher apparent L-value, which is a lighter (less dark) appearance, while the more textured quench side shows a lower apparent L-value, which is a darker color. Because of this observation, it is possible to select the outward-facing surface of the membrane for use in such a manner as would best benefit the particular application utilizing the composite microarray slide of the present disclosure, either by surface texture, or by color, or both.

Alternately, if additional manipulations in color from side to side are desired, alternative casting methods and products such as that described in U.S. Pat. No. 6,513,666 may be employed to achieve either color symmetry or asymmetry, as would be known to those skilled in the art. Additionally, the same methods and products described in U.S. Pat. No. 6,513,666 may also be employed to produce either symmetric, asymmetric, or other multi-zone membranes with respect to pore size, as would be known to those skilled in the art.

Six slides of the drum side outward facing membrane surface were selected for further testing. Compared to the white slides of the previous Example, the gray slides of the present Example show acceptable aesthetic properties, with even color distribution across surface of slide, with an average L-value of 58 units.

As described in co-pending commonly owned application Ser. Nos. 09/897,333, and 09/899,607 already incorporated herein by reference, fluorescent background (autofluorescence) from the support material upon which nucleic acids are spotted is detrimental to the sensitivity of fluorescent detection technology on the array. Efficiency of the hybridization signal across the array can be affected by inconsistent background on the array, which reduces dynamic range and increases the coefficient of variation of signal ratios on DNA microarrays and makes detection of genes expressed at low levels problematic.

Similarly, when chemiluminescence is employed as the preferred method for detecting hybridization events, non-specific chemiluminescence from the substrate and more importantly reflectance from specific signals on the array reduce sensitivity, dynamic range, while increasing coefficients of variation among signal ratios on the array. As such, reflectance can negate the ability to differentiate slight differences in signal among genes. In addition, the reflectance generated from intense signals (features with high gene expression levels) can obscure neighboring features. Further, distribution of intense signals across the array surface can result in overall background noise from the light emitted from the features; therefore, reduction of reflectance is a desirable attribute for an array platform. The pigmented nylon membrane composite microarray slides described herein are very effective in reducing background light emission from chemiluminescent signals.

As can be seen above, these Examples demonstrate that composite microarray slides useful for carrying a microarray of biological polymers on the surface thereof has been produced using a wet-as-cast nylon membrane and a glass substrate by treating the glass substrate with chemical agents which may include a polymeric intermediate layer as a surface treatment to produce an attachment layer that facilitates the covalent or other type of bonding between the wet-as-cast nylon membrane and the glass substrate in such a manner as to be useful in microarray applications.

As would be a understood by those skilled in the art, the above examples are merely representative of a plurality of possible examples that could be prepared in accordance with the concepts taught by the present disclosure.

Similarly, it is further understood that deviations within the specific mechanisms of the Examples above would be understood by those skilled in the art without the necessity of a production blueprint.

As illustrated in Table 2, below, the survivability of the improved composite microarray slides of the present disclosure, when compared with competitive slides is clearly demonstrated. As detailed in Table 1, survivability factors were assigned values according to the relative delamination of the membrane. In all conditions depicted below, the slides were placed, two (2) at a time, into a 60 mL beaker filled with the desired solution and then sealed. Three (3) slides of each condition were then placed into a hybridization oven and allowed to equilibrate to the desired temperature, for the desired exposure time. Following exposure, the slides were removed from the desired solution, and rubbed firmly by an operator with a gloved finger in an effort to simulate a worst-case evaluation similar to the effect of agitation, follow-up wash steps, or other customer-performed protocols, on the surface of the slides, after treatment with the desired solution.

The slides were then evaluated according to a “Survivability Factor,” as described in Table 1 below:

TABLE 1

| Value | Description |
|---|---|
| 1 | Membrane floating in solution, no attachment |
| 2 | Membrane detaches readily from slide with slight rubbing |
| 2.5 | Membrane detaches from slide in one continuous piece, but still |
| | has some attachment to glass |
| 2.75 | Small portions of membrane are fixed to glass, with the balance |
| | delaminating easily, or it takes more force to separate membrane |
| | from glass than 2.5. |
| 3 | Membrane delaminates in small pieces when rubbed firmly. |
| 3.25 | Significant delamination across surfaceof slide, but with at least |
| | ⅔ of glass still fixed |
| 3.5 | Delamination between approximately 5% and 20% of the slide area |
| 3.75 | Delamination in small areas, generally corners, no more than 5% of |
| | slide area |
| 4 | No delamination |

During the evaluations, the slides produced in Example 1 above were not tested, since the slides produced in Example 4 used the identical chemistry to produce the attachment layer, as those in Example 1. As can be seen from Table 2 below, the competitive nylon-glass composites that were tested all exhibited substantial bond strength weakening or delamination after exposure to the various hostile environments. Thus, Table 2 below indicates that the competitive slides are not stable for use in these hostile environments, and, are believed likely to encounter problems with delamination in the field during critical commercial operations.

TABLE 2

| | | | Survivability Rating (0-4, higher = better) | | | | | | | | | | | | | | |
|---|---|---|---|---|---|---|---|---|---|---|---|---|---|---|---|---|---|
| | | | | | | Example 3 | | | Example 4 | | | | | | | | |
| | | Exposure | Example 2 | | | (Adcote | | | (Gray | | | | | | S&S CAST | | |
| Solution | Temp | (hr) | (828/3115) | | | 89R3) | | | 828/3115) | | | Pall Vivid Slides | | | slides | | |
| 4X SSC | 60 | 15 | 4 | 4 | 4 | 4 | 4 | 4 | 4 | 4 | 4 | 2.75 | 2.75 | 3 | 4 | 2.5 | 2.5 |
| 4X SSC | 85 | 4 | 4 | 4 | 4 | 4 | 3.5 | 2 | 4 | 4 | 4 | 2.75 | 2.75 | 2.75 | 2.75 | 3 | — |
| Rosetta | 60 | 15 | 4 | 3.75 | 3.75 | 4 | 4 | 3.8 | 4 | 4 | 4 | 2.75 | 2.75 | 2.75 | 2 | 2.5 | 2.5 |
| DMF | RT | 6 | 4 | 4 | 4 | 2 | 2 | 2 | 4 | 4 | 4 | 2.5 | 2.5 | 2.5 | 4 | 4 | 4 |
| DMSO | RT | 6 | 4 | 4 | 4 | 2.5 | 2.5 | 2.8 | 4 | 4 | 4 | 2.5 | 2.5 | 2.5 | — | — | — |
| 1% SDS | Boiling | 10 min. | 4 | 4 | 4 | 4 | 4 | 3.5 | 4 | 4 | 4 | 2.5 | 2.5 | 2.5 | 1 | 1 | 1 |

Note:
— = Not tested (insufficient # of samples to test this condition)
RT = room temperature

The solutions used in Table 2 above to test for chemical compatibility are defined in accordance with the following:

4×SSC (sodium citrate, sodium chloride, Aldrich #93017), is a frequently used solution for hybridization and washing of microarrays and nylon membranes.

Rosetta is a known hybridization solution, the protocol for synthesis is describe in Nature Biotechnology, 2001 Vol 19, pgs 342-34, the disclosure of which is herein incorporated by reference to the extent not inconsistent with the present disclosure.

DMF, (n-dimethylformamide, Sigma-Aldrich #22,705-6), is a common organic solvent.

DMSO, (dimethyl sulfoxide, Sigma-Aldrich #D1435), is another common organic solvent frequently used in biological applications.

1 wt % SDS, (sodium dodecyl sulfate), is a surfactant solution frequently used to wet nylon membranes.

The improved composite microarray slides of the present disclosure, when tested using the various hostile environments, clearly demonstrated sufficiently strong attachment in order to survive in the target hostile environments. In nearly all cases, the improved composite microarray slides of the present disclosure, exhibited little, if any, weakening of the bond strength between the substrate and the microporous membrane even with very firm rubbing, with the only significant exception being Example 3, which uses an alternative linker that is known to be incompatible in organic solvents such as DMF and DMSO. However, the attachment bond strength remained stable in the other solvents tested, as is evident from Table 2.

Thus, it appears evident from the above data that the improved composite microarray slides of the present disclosure, when produced utilizing the representative and preferred surface treatments to prepare the substrate for bonding to the microporous membrane results in an attachment layer that should definitively prevent delamination of the substrate from the membrane during customer testing, even with vigorous wash cycles after treatment in the desired solution and, more significantly, during actual commercial applications using the improved composite microarray slides of the present disclosure.

Composite microarray slides from Example 4 were next tested for fluorescence background. Since the base structure of the nylon microporous membrane was not changed by the addition of carbon black, it was theorized that any reduction in the fluorescence between the nylon membrane of Examples 1 and 4 would be due solely to adsorption of fluorescent signal by the carbon black. It was further expected that fluorescence and reflectance (in chemiluminescent assays) will be related, and that a black material which is known to adsorb light in common fluorescent wavelengths (about 500-700 nm), would be expected to adsorb light in at least the entire visible light region (about 400 to about 700 nm), including the region typically used for chemiluminescence (around 460 nm).

For this test, three slides each from Examples 1 and 4 above (which differed only in that carbon black was added to the membrane in Example 4) were scanned for fluorescent background using an Axon Genepix 4000B scanner. This laser-based device bombarded the slide with light at a precisely fixed excitation wavelength, and then measured the intensity of the response at the frequencies described above in “fluorescence units.” Five points per slide were selected and then measured for the “fluorescence units,” and the total averaged for each wavelength. The test were conducted using a machine setting of 33% power and a 600 photomultiplier tube (PMT).

TABLE 3
BACKGROUND AND REFLECTANCE

| | | Fluorescence (Axon units) | |
|---|---|---|---|
| Example | Membrane Type | 635 nm | 532 nm |
| 1 | White | 420 ± 90 | 17000 ± 3000 |
| 4 | Gray (L = 58) | 70 ± 10 | 1200 ± 400 |

*Error bars are ±1 standard deviation

As shown in Table 3, the composite microarray slides of Example 4 demonstrated substantially reduced background fluorescence by the addition of carbon black to the membrane. It is believed that this substantially reduced background fluorescence would be strongly indicative of corresponding reductions in the reflectivity of the membrane, as would be expected by one skilled in the art.

In summary, several variations of the representative, presently preferred method of fabricating composite microarray slides have resulted in permanent and robust bonding of the microporous membrane, specifically nylon microporous membrane, to various solid substrates, capable of withstanding the rigors of microarray applications, having the advantages of a thin and uniform functional nylon layer of useful for binding biomolecular analytes of DNA and other genomic products, proteins, etc., and suffering none of the drawbacks of the currently available microarray glass slides. Such drawbacks include, but are not limited to, having no membrane at all (functionalized glass), or the use of nitrocellulose as a less preferred membrane, or the use of a reinforced nylon membrane having variable thickness characteristics that exceed the presently required tolerance, or the use of an adhesive layer between the membrane and the glass substrate that is not capable of withstanding the rigors of microarray applications.

As can be clearly seen above, in accordance with the present disclosure, representative microporous membrane, specifically nylon membrane, was effectively bound to a substrate, such as, for example, a glass slide, with a surface treatment selected from chemical agents that produced bonding between the nylon and the substrate resulting in an attachment layer that proved to be resilient/survivable in hybridization conditions and commercial solvents used in the intended environment to form the composite microarray slides. Competitive nylon-glass slides all are believed to delaminate under these conditions, as demonstrated in Table 2 above.

The chemical agents that comprise surface treatments of the present disclosure result in a thin, uniform nylon-slide attachment layer (approx. 1-2 microns) thick, which is much thinner than most commercial adhesives (typically 10-25 microns minimum), formed from the chemical anchor and linker associations that comprise the surface treatment placed between the nylon and the substrate. The combination of the nylon/surface treatment/substrate has been found to reduce variability in flatness, and also to lessen the possibility of harmful interference or chemical reactivity between various substrates used in the end use applications and chemical agents that comprise the surface treatments that result in the attachment layer.

In addition to the properties described above, the composite microarray slides of the present disclosure display a uniformity in flatness and thickness, most likely due to well controlled attachment layer thickness and the tendency of the swollen, wet, structure of undried microporous phase-inversion nylon to shrink and physically conform to an underlying substrate such as glass during restrained drying.

Further, with the composite microarray of the present disclosure, there is no need for a costly drying step before application of membrane to glass, which has been found to add “belt lines” and other aesthetic defects.

Composite microarray slides having components made by the phase inversion process and especially nylon membrane bound to a polymer substrate instead of glass have many potential microarray applications. The following is an attempt to describe representative processes for the production of such representative composite microarray slides having a porous membrane formed by a phase inversion process operatively attached by, presently preferably, covalent bonding through a surface treatment, presently preferably, a polymeric intermediate layer resulting in an attachment layer to a polymer substrate such that the combination produced thereby is useful in microarray applications.

The following representative prophetic Examples describe the steps that are believed necessary to produce nylon/non-porous support material composites other than the nylon/glass and composites that have been made (as described in Examples 1-4 above). The nylon/non-porous support material composites made would contain a thin (about 4 mil or less) porous nylon membrane bound to the surface of a non-porous support material.

As might be anticipated, different non-porous support materials must be pre-treated in different ways. The following describes the pre-treatments for different non-porous support materials believed to have utility in the subject matter of the present disclosure:

1) Ceramic non-porous support material: Mix about 95 mL of ethanol, about 5 mL of water, and about 2 mL of 3-aminopropyl trimethoxysilane and let stand for about five minutes. Submerge the substrate into the solution for about two minutes, remove and rinse with ethanol. Heat the substrate for about 10 minutes at about 120° C., and let sit overnight. This particular solution should produce a considerable number of bonding sites for the linker chemistries and nylon to the ceramic non-porous support material.

2) Acrylic non-porous support material: Acrylic polymers (acrylonitriles) contain nitrite bonds at most repeat units (not every repeat unit, as they tend to copolymerize). To prepare such support material for bonding with nylon, hydrolyze the nitrites to carboxylic acid groups by submerging the substrate in about 5M HCl (acid or base catalyzes the reaction) for about 10 minutes. This particular solution will produce a great number of bonding sites for the linker and nylon to the acrylic polymers.

3) Polypropylene non-porous support material: Polypropylene is a relatively unreactive material. To make polypropylene open for bonding, treat the surface of the polypropylene with about a 0.4 KW corona discharge. It is believed that the corona discharge may free up some bonding sites by producing carboxylic acid groups and carbonyl groups on the surface of the polypropylene non-porous support material. Because the effects of corona treatment may wear off over time, it is believed best to proceed to the next step, as described below, immediately. Alternatively, plasma treatment could also be used to introduce carboxyl or carbonyl groups into the surface which are suitable for bonding. Suitable gases for treatment may include helium, oxygen, acetylene, and carbon dioxide.

4) Polycarbonate and Polysulfone non-porous support material: The Polycarbonate and Polysulfone non-porous support material is placed in aqueous solution of about 1M NaOH with a bromine substituted carboxylic acid such as, for example, bromoacetic acid. The bromoacetic acid condenses with the phenol end groups of the polymer, releasing HBr as a side product. The resultant product of the condensation reaction has chains that now end with a carboxylic acid group that can then bond with the linker and nylon.

5) Polyamide and Polyaramid non-porous support material: These polymers already contain carboxylic acid and amine end groups that can be used to react in the next step. They are presently believed not to require a pre-treatment.

Following the appropriate pretreatment as described above, an epoxy solution is prepared using the following components:

about 10 grams Epon 828 (a Bisphenol A type epoxy resin); and

about 35 grams Xylene.

In a separate 250 mL Erlenmeyer flask, the following were also added:

about 6 grams Epikure 3125 (a polyamide based curing agent);

about 35 grams Xylene; and

about 1.8 grams 3-glycidopropyltrimethoxysilane.

The resulting solution is then mixed for about 5 hrs at about 60° C., and then applied to the surface of the appropriate non-porous support materials as a representative surface treatment via spin coating or any other means, as would be understood by one skilled in the art.

The epoxy group on the Bisphenol A molecule should bond with the amino or carboxylic acid groups on the respective non-porous support material and the amino and carboxylic acid groups on the nylon, thereby bonding the nylon and the respective non-porous support material together producing a attachment layer therebetween.

After the respective non-porous support material is pre-treated as described above, wet-as-cast porous nylon membrane (as described in U.S. Pat. Nos. 3,876,738 and 4,707,265) is placed over the respective non-porous support material and the wet-as-cast porous nylon membrane is stretched. Personnel wearing gloves only handle the wet-as-cast porous nylon membrane. The wet-as-cast porous nylon membrane is obtained for applying to the respective non-porous support material after the wet-as-cast porous nylon membrane is cast, quenched, and washed with DI water, but has not yet been exposed to a drying step, hence the term “wet-as-cast.” The type of polymer used is presently preferably a high molecular weight, high amine nylon.

Care is taken to remove any air bubbles between the wet-as-cast porous nylon membrane and the respective non-porous support material. The wet-as-cast porous nylon membrane is flattened on the respective non-porous support material and all wrinkles are removed from the wet-as-cast porous nylon membrane/respective (unclear) non-porous support material combination. The wet-as-cast porous nylon membrane is then clipped into place on a hemi-drum. The entire assembly is heated in a convection oven at about 110° C. for about one hour. After heating, the excess porous nylon membrane is removed from the respective non-porous support material by cutting away the edges of the porous nylon membrane from the respective non-porous support material, as is known in the art.

The resulting porous nylon membrane/respective non-porous support material composites should have a very thin, smooth layer of porous nylon membrane operatively bound to the respective non-porous support material via an attachment layer. The porous nylon membrane surface should be free of deformities, marks or particles.

When tested in DI water, about 0.4M sodium hydroxide, and about 1% sodium dodecyl sulfate (SDS) in water, the nylon should wet readily. The bond between the porous nylon membrane component and the respective non-porous support material component of the resulting porous nylon membrane/respective non-porous support material composites should exhibit strong bonding, and the porous nylon membrane component should not peel away or delaminate from the respective non-porous support material component.

The bond between the porous nylon membrane component and the respective non-porous support material component should stay strong even when the resulting porous nylon membrane/respective non-porous support material composites are quickly submerged vertically into boiling solutions of water or SDS. Despite the harshness of this treatment, the uncharged resulting porous nylon membrane/respective non-porous support material composites should retain their peel strength, i.e., the porous nylon membrane component should rip before peeling away or delaminating from the respective non-porous support material component.

The above representative prophetic Examples are based on accepted principles of the synthesis of the various substrates (inorganic or organic polymers) and their surface reactivities, regarding the preparation of the surfaces for receptivity to the bifunctional linking chemistries as disclosed in the present disclosure. These accepted principles of synthesis are not meant to be limitations on the preparations of the respective non-porous support material component. The accepted principles of synthesis are merely suggestions for defining starting points in the practice of the present disclosure, and may be modified by one skilled in the art, but still be in accordance with the inventive teachings of the present disclosure.

As should be clear from the above Examples and other description, the following specific chemicals have been found effective as the anchor surface treatment component, silane surface “anchors”: 3-aminopropyl triethoxysilane, N-(2-aminoethyl)-3-aminopropyl trimethoxysilane, 3-glycidoxypropyltrimethoxysilane, and 3-aminopropyldimethylethoxysilane.

The following specific representative chemicals have been found effective as the linker surface treatment component (“linkers”): in general, an epoxy functional long-chain polymer, particularly, Bisphenol A, more specifically, Epon 828, made by Resolution Performance Products. Additionally, a polyester-silane type polymer, known commercially as Adcote 89R3 polymer, made by Rohm and Haas, has been found effective.

As should be apparent to those skilled in the art, nylon is the presently preferred substrate of use in nucleic acid detection assays. The reason that nylon is presently preferred over nitrocellulose is that nylon has a higher intrinsic positive charge. It is generally recognized that nylon, with its peptide backbone linkage, and well-defined end-group chemistries, provides charge interactions which nitrocellulose cannot provide. Biomolecule binding to nitrocellulose is dependent primarily on hydrophobic interactions. Biomolecule binding in nylon is believed to be a function of charge. Additionally, nylon can be charged modified, thereby increasing the binding capacity of the nylon for nucleic acid. Also, nylon is much more robust than nitrocellulose, does not easily break, can be stripped and reprobed, is not an extreme fire hazard like nitrocellulose and is amenable to much more stringent washing and hybridization conditions.

The anchor and linker components of the chemical agents comprising the surface treatment of the present disclosure produces an attachment layer having minimal discernable finite thickness or mass that could add nonuniformity to the overall thickness of the substrate/membrane combination structure and does not participate in the binding or detection of nucleic acid or protein analytes. This eliminates possible physical interference from the presence of an adhesive layer by precluding nonuniformity in thickness, and eliminates possible chemical interference by the absence of an additional substance that could participate in chemical reactions.

In view of the above described Examples and in accordance with the present disclosure, the following generic definitions for the various chemical agents comprising the components of the surface treatment that produces the attachment layer are believed representative of the specific chemical agents useful in the particular applications and for the specific purposes described herein.

Anchors:

Organosilanes of the presently preferred representative embodiments according to the present disclosure have the following structures:

SiR¹X₃ 1.

SiR¹XA₂ 2.

SiR¹X₂A 3.

wherein R¹ is an alkyl, substituted alkyl, cycloalkyl, alkenyl, or alkynyl group; each bearing a terminal functional group, R² wherein R² is olefin, vinyl, acrylate, methacrylate, or allyl amino group; an alkyl-hydroxyl, aldehyde, keto, halo, acylhalide, or carboxyl group; aryloxy, alkanoyloxy, amino, alkylamino, arylamino, aralkylamino, cycloalkylamino, heterocycloamino, disubstituted amines, alkanoylamino, aroylamino, aralkanoylamino, thiol, alkylthio, arylthio, cycloalkylthio, heterocyclothio, alkylthiono, arylthiono, alkylsulfonyl, arylsulfonyl, aralklsulfonyl, sulfonamido, substituted sulfonamido, nitro, cyano, carboxy, carbamyl, substituted carbamyl, alkoxycarbonyl, or epoxy.

Examples of R¹ include but are not limited to 3-aminopropyl, 3-aminopropylmethyl, N-(2-aminoethyl)-3-aminopropylmethyl, aminophenyl, 4-aminobutyldimethyl, aminoethylaminomethyphenethyl, or mixtures thereof.

Specific examples of the silane include: 3-glycidoxypropyltrimethoxysilane, 3-aminopropyl triethoxysilane, 3-aminopropylmethyldiethoxysilane, 3-aminopropyl dimethylethoxysilane, 3-aminopropyl trimethoxysilane, N-(2-aminoethyl)-3-aminopropylmethyl dimethoxysilane, N-(2-aminoethyl-3-aminopropyl) trimethoxysilane, aminophenyl trimethoxysilane, 4-aminobutyldimethyl methoxysilane, 4-aminobutyl triethoxysilane, aminoethylaminomethyphenethyl trimethoxysilane, or mixtures thereof. Also, 3-(trimethoxysilyl)propyl methacrylate, N-[3-(trimethoxysilyl)propyl]-N′-(4-vinylbenzyl)ethylenediamine, triethoxyvinylsilane, triethylvinylsilane, vinyltrichlorosilane, vinyltrimethoxysilane, and vinylytrimethylsilane.

A is any alkyl, ether, halide, R⁵—O—, and/or R⁶—O—, wherein R⁵ and R⁶ are independently hydrogen, alkyl, alkenyl, alkynyl, cycloalkyl, cycloalkenyl, aryl, or heterocyclo. Examples include, but are not limited to methoxyl, ethoxyl, methyl, ethyl, propyl, butyl, ethylvinyl, trichloromethyl, trifluoromethyl, trifluoromethoxy, trichloromethoxy, methylvinyl, chloro, ethoxyvinyl, vinyltrichloro, vinyltrimethoxy, vinylytrimethyl, and mixtures thereof.

X is a hydrolyzable group capable of condensation on a glass surface, including hydroxy, alkoxy, cyloalkoxy, heterocyclooxy, oxo, alkanoyl, aryloxy, alkanoyloxy, R⁵—O—, and/or R⁶—O—, wherein R⁵ and R⁶ are independently hydrogen, alkyl, alkenyl, alkynyl, cycloalkyl, cycloalkenyl, aryl, or heterocyclo groups.

Linker Molecules:

Linker molecules suitable for use in the preferred embodiment include any cross-linkable molecule bearing a functional group that is capable of binding to nylon. Suitable molecules include, but are not limited to: Bisphenol “A” also, acrylic acid, methacrylic acid, vinylacetic acid, 4-vinylbenzoic acid, itaconic acid, allyl amine, allylethylamine, 4-aminostyrene, 2-aminoethyl methacrylate, chlorostyrene, dichlorostyrene, 4-hydroxystyrene, hydroxymethyl styrene, vinylbenzyl alcohol, allyl alcohol, 2-hydroxyethyl methacrylate, poly(ethylene glycol) methacrylate, methyl acrylate, methyl methacrylate, ethyl acrylate, ethyl methacrylate, styrene, 1-vinylimidazole, 2-vinylpyridine, 4-vinylpyridine, divinylbenzene, ethylene glycol dimethacryarylate, N,N′-methylenediacrylamide, N,N′-phenylenediacrylamide, 3,5-bis(acryloylamido)benzoic acid, pentaerythritol triacrylate, trimethylolpropane trimethacrylate, pentaerythritol tetraacrylate, trimethylolpropane ethoxylate (14/3 EO/OH) triacrylate, trimethyolpropane ethoxylate (7/3 EO/OH) triacrylate, triethylolpropane propoxylate (1 PO/OH) triacrylate, trimethyolpropane propoxylate (2 PO/PH triacrylate), or polyesters (saturated and unsaturated).

Specific examples of suitable linkers include Epon 828 (a Bisphenol A diglycidyl ether), available commercially from Resolution Performance Products, and Adcote 89R3, a polyester-silane commercially available from Rohm and Haas.

Cross linkers:

Suitable molecules for crosslinking include any molecule containing at least two functional groups that are capable of bonding to the linker. The nonfunctionalized chain length extension portions of the molecule (or “backbone”) may include monomers or n-mers capable of polymerization such as polymethylmethacrylate (PMMA), polycarbonate, polyvinylchloride (PVC), polydimethylsiloxane (PDMS), polysulfone, polystyrene, polymethylpentene, polypropylene, polyethylene, polyvinylidine fluoride, ABS (acrylonitrilebutadiene-styrene copolymer), and the like. Moreover, crosslinking molecules may also perform the function of a secondary linker, whereby the cross linker binds to the intended linker and to the nylon.

Additional “backbones” include any aliphatic or aromatic molecule which could contain a repeating functional group that will bind with the target linker molecule. Suitable functional groups are selected from (but not limited to): acrylate, methacrylate, or allyl amino group; an alkyl-hydroxyl, aldehyde, keto, halo, acylhalide, or carboxyl group; aryloxy, alkanoyloxy, amino, alkylamino, arylamino, aralkylamino, cycloalkylamino, heterocycloamino, disubstituted amines, alkanoylamino, aroylamino, aralkanoylamino, thiol, alkylthio, arylthio, cycloalkylthio, heterocyclothio, alkylthiono, arylthiono, alkylsulfonyl, arylsulfonyl, aralklsulfonyl, sulfonamido, substituted sulfonamido, nitro, cyano, carboxy, carbamyl, substituted carbamyl, alkoxycarbonyl, or epoxy.

Specific examples include commercially available cross linkers such as Epikure 3125, 3115, and W50, from Resolution Performance Products, Inc, and tetraethylenepentamine (Dow Corp).

As taught in the broadest interpretation possible by the present disclosure, it should be clear that one who is skilled in the art could readily determine how to anchor a different bifunctional silane into the glass, or a bifunctional reactive polymer into the glass (or other solid substrate), and use the opposite end to link either directly or through an intermediate, to any porous polymer membrane which has linkable functional groups and all such operable combinations are believed taught by the present disclosure in a manner sufficient for one skilled in the art to accomplish same without undue experimentation.

While experiments have not as yet been conducted to verify that the same or similar results when using the other chemical agents, of the present disclosure, as anchors and linkers, it is presently believed that the other chemical agents of the present disclosure can be useful in the processing of a large number of surface treatments to produce composite microarray slides for useful purposes because of the similar chemical compositions, compatible functional groups, and structures of the disclosed chemical agents.

Thus, it should be apparent from the above that the present disclosure has provided improved composite microarray slides useful for carrying a microarray of biological polymers on the surface thereof and, more particularly, to an improved composite microarray slide having a porous membrane formed by a phase inversion process effectively attached by covalent bonding or hydrogen bonding through chemical agents that comprise a surface treatment to a substrate, the surface treatment preparing the substrate to sufficiently bond to the microporous membrane through the attachment layer formed therebetween resulting from the surface treatment such that the combination produced thereby is useful in microarray applications. Specifically, the improved composite microarray slides of the present disclosure comprise porous media and a substrate and that are bound by a surface treatment that comprise chemical agents which results in the formation of an attachment layer that overcame the primary functional problem of survivability of the nylon-glass slide in various test solutions, particularly hybridization solutions, such as, for example, 4×SSC. Additionally, solvent resistance (such as DMF) was also overcome as was the problem of maintaining acceptable aesthetic properties and uniformity. Moreover, pigmented membranes result in reduced reflectance and reduced fluorescence.

While the articles, apparatus and methods for making the articles contained herein constitute preferred embodiments of the invention, it is to be understood that the disclosure is not limited to these precise articles, apparatus and methods, and that changes may be made therein without departing from the scope of the disclosure which is defined in the appended claims.

What is claimed is:

1. A composite microarray slide, useful for carrying a microarray of biological polymers comprising:
a microporous membrane formed by a phase inversion process;
a non-porous substrate; and
an attachment layer, the attachment layer comprising at least one anchor and at least one linker, the attachment layer being operatively positioned between the microporous membrane and the non-porous substrate, the attachment layer sufficiently bonding the non-porous substrate to the microporous membrane such that the combination composite microarray slide is useful in microarray applications.

2. The composite microarray slide of claim 1, wherein the attachment layer is between about 0.1 to about microns thick.

3. The composite microarray slide of claim 1, wherein the attachment layer is between about 2 to about 5 microns thick.

4. The composite microarray slide of claim 1, wherein the attachment layer is about 3 microns thick.

5. The composite microarray slide of claim 1, wherein the attachment layer has a uniform thickness.

6. The composite microarray slide of claim 1 wherein the attachment layer has minimal finite thickness or mass.

7. The composite microarray slide of claim 1 wherein the attachment layer at least substantially eliminates nonuniformity in the overall thickness of the composite microarray slide.

8. The composite microarray slide of claim 1 wherein the microporous membrane further comprises:
a sufficient amount of pigments.

9. The composite microarray slide of claim 8 wherein the pigments comprise:
carbon-black.

10. The composite microarray slide of claim 8 wherein when compared to a microarray slide with a microporous membrane containing no pigments, substantially reduced fluorescence is observed.

11. The composite microarray slide of claim 8 wherein when compared to a microarray slide with a microporous membrane containing no pigments, substantially reduced reflectance is observed.

12. The composite microarray slide of claim 1 wherein the microporous membrane is asymmetric.

13. The composite microarray slide of claim 1 wherein the microporous membrane is symmetric.

14. The composite microarray slide of claim 1 wherein the attachment layer covalently bonds the non-porous substrate and the microporous membrane.

15. The composite microarray slide of claim 1 wherein the presence of the attachment layer results in minimal interference in the binding of the biological polymer.

16. The composite microarray slide of claim 1 wherein the presence of the attachment layer results in minimal interference in the detection of the biological polymer.

17. The composite microarray slide of claim 15 wherein the biological polymer comprises:
a nucleic acid.

18. The composite microarray slide of claim 15 wherein the biological polymer comprises:
a protein.

19. The composite microarray slide of claim 15 wherein the biological polymer comprises:
a peptide.

20. The composite microarray slide of claim 15 wherein the biological polymer comprises:
an enzyme.

21. The composite microarray slide of claim 15 wherein the biological polymer comprises:
an antibody.

22. The composite microarray slide of claim 16 wherein the biological polymer comprises:
a nucleic acid.

23. The composite microarray slide of claim 16 wherein the biological polymer comprises:
a protein.

24. The composite microarray slide of claim 16 wherein the biological polymer comprises:
a peptide.

25. The composite microarray slide of claim 16 wherein the biological polymer comprises:
an enzyme.

26. The composite microarray slide of claim 16 wherein the biological polymer comprises:
an antibody.

27. The composite microarray slide of claim 1 wherein when subjected to an organic solvent system for greater than about 6 hours, the microporous membrane does not delaminate significantly from the non-porous substrate.

28. The composite microarray slide of claim 1 wherein when subjected to 4×SSC at about 60° C. for greater than about 10 hours, the microporous membrane does not delaminate significantly from the non-porous substrate.

29. The composite microarray slide of claim 1 wherein when subjected to 4×SSC at about 60° C. for about 2 weeks, the microporous membranes does not delaminate significantly from the non-porous substrate.

30. The composite microarray slide of claim 1 wherein, the attachment layer comprises:
an organosilane, operatively reacted with a polyamido-polyamine epichlorohydrin resin.

31. The composite microarray slide of claim 30, wherein the organosilane is selected from the group comprising:
3-aminopropyltriethoxysilane, 3-aminopropyldimethyethoxysilane, 3-lycidopropyltrimethoxysilane or equivalents thereof.

32. The composite microarray slide of claim 1 wherein, the at least one anchor comprises:
an organosilane, SiR¹X₃
wherein R¹ is an alkyl, substituted alkyl, cycloalkyl, alkenyl, or alkynyl group; each bearing a terminal functional group, wherein the terminal functional group is olefin, vinyl, acrylate, methacrylate, or allyl amino group; an alkyl-hydroxyl, aldehyde, keto, halo, acylhalide, or carboxyl group; aryloxy, alkanoyloxy, amino, alkylamino, arylamino, aralkylamino, cycloalkylamino, heterocycloamino, disubstituted amines, alkanoylamino, aroylamino, aralkanoylamino, thiol, alkylthio, arylthio, cycloalkylthio, heterocyclothio, alkylthiono, arylthiono, alkylsulfonyl, arylsulfonyl, aralklsulfonyl, sulfonamido, substituted sulfonamido, nitro, cyano, carboxy, carbamyl, substituted carbamyl, alkoxycarbonyl, or epoxy; and
X is selected from the group consisting of hydroxy, alkoxy, cyloalkoxy, heterocyclooxy, oxo, alkanoyl, aryloxy, alkanoyloxy, trifluoromethyl, trifluoromethoxy, hydrogen, alkyl, R⁵—O—, and/or R⁶—O—, wherein R⁵ and R⁶ are independently hydrogen, alkyl, alkenyl, alkynyl, cycloalkyl, cycloalkenyl, aryl, heterocyclo or equivalents thereof.

33. The composite microarray slide of claim 1 wherein, the at least one anchor comprises:
an organosilane, SiR¹XₘAₙ
wherein m is either 1 or 2, n is either 1 or 2;
R¹ is an alkyl, substituted alkyl, cycloalkyl, alkenyl, or alkynyl group; each bearing a terminal functional group, wherein the terminal functional group is olefin, vinyl, acrylate, methacrylate, or allyl amino group; an alkyl-hydroxyl, aldehyde, keto, halo, acylhalide, carboxyl group; aryloxy, alkanoyloxy, amino, alkylamino, arylamino, aralkylamino, cycloalkylamino, heterocycloamino, disubstituted amines, alkanoylamino, aroylamino, aralkanoylamino, thiol, alkylthio, arylthio, cycloalkylthio, heterocyclothio, alkylthiono, arylthiono, alkylsulfonyl, arylsulfonyl, aralklsulfonyl, sulfonamido, substituted sulfonamido, nitro, cyano, carboxy, carbamyl, substituted carbamyl, alkoxycarbonyl, or epoxy;
A is selected from the group consisting of alkyl, ether, halide, R⁵—O—, and/or R⁶—O—, wherein R⁵ and R⁶ are independently hydrogen, alkyl, alkenyl, alkynyl, cycloalkyl, cycloalkenyl, aryl, or heterocyclo; and
X includes hydroxy, alkoxy, cyloalkoxy, heterocyclooxy, oxo, alkanoyl, aryloxy, alkanoyloxy, trifluoromethyl, trifluoromethoxy, R⁵—O—, and/or R⁶—O—, wherein R⁵ and R⁶ are independently hydrogen, alkyl, alkenyl, alkynyl, cycloalkyl, cycloalkenyl, aryl, heterocyclo or equivalents thereof.

34. The composite microarray slide of claim 1, wherein the at least one anchor comprises an organosilane, the organosilane selected from the group comprising:
3-aminopropyltriethoxysilane, 3-aminopropyldimethyethoxysilane and 3-glycidopropyltrimethoxysilane.

35. The composite microarray slide of claim 1 wherein, the linker comprises:
a polymer comprising at least one functional group that is capable of binding to the anchor and at least one functional group that is capable of binding to the nylon.

36. The composite microarray slide of claim 35 wherein, the linker further comprises:
saturated polyesters or unsaturated polyesters

37. The composite microarray slide of claim 35 wherein, the linker is selected from the group comprising:
Bisphenol “A”, Adcote 89R3, acrylic acid, methacrylic acid, vinylacetic acid, 4-vinylbenzoic acid, itaconic acid, allyl amine, allylethylamine, 4-aminostyrene, 2-aminoethyl methacrylate, chlorostyrene, dichlorostyrene, 4-hydroxystyrene, hydroxymethyl styrene, vinylbenzyl alcohol, allyl alcohol, 2-hydroxyethyl methacrylate, poly(ethylene glycol) methacrylate, methyl acrylate, methyl methacrylate, ethyl acrylate, ethyl methacrylate, styrene, 1-vinylimidazole, 2-vinylpyridine, 4-vinylpyridine, divinylbenzene, ethylene glycol dimethacryarylate, N,N′-methylenediacrylamide, N,N′-phenylenediacrylamide, 3,5-bis(acryloylamido)benzoic acid, pentaerythritol triacrylate, trimethylolpropane trimethacrylate, pentaerythritol tetraacrylate, trimethylolpropane ethoxylate (14/3 EO/OH) triacrylate, trimethyolpropane ethoxylate (7/3 EO/OH) triacrylate, triethylolpropane propoxylate (1 PO/OH) triacrylate, or trimethyolpropane propoxylate (2 PO/PH triacrylate).

38. The composite microarray slide of claim 35, wherein the linker comprises:
Bisphenol “A”.

39. The composite microarray slide of claim 35, wherein the linker comprises:
Adcote 89R3.

40. The composite microarray slide of claim 1 wherein, the attachment layer further comprises:
a cross linker.

41. The composite microarray slide of claim 40 wherein, the cross linker further comprises:
a backbone and at least two functional group.

42. The composite microarray slide of claim 40 wherein, the backbone of the cross linker further comprises:
an aliphatic or an aromatic moiety which contains at least two functional group that will bind to the linker molecule.

43. The composite microarray slide of claim 41 wherein, the backbone of the cross linker is selected from the group comprising:
polymethylmethacrylate (PMMA), polycarbonate, polyvinylchloride (PVC), polydimethylsiloxane (PDMS), polysulfone, polystyrene, 5 polymethylpentene, polypropylene, polyethylene, polyvinylidine fluoride, ABS (acrylonitrilebutadiene-styrene copolymer), or equivalents.

44. The composite microarray slide of claim 42 wherein the functional group is selected from the group comprising:
acrylate, methacrylate, or allyl amino group; an alkyl-hydroxyl, aldehyde, keto, halo, acylhalide, or carboxyl group; aryloxy, alkanoyloxy, amino, alkylamino, arylamino, aralkylamino, cycloalkylamino, heterocycloamino, disubstituted amines, alkanoylamino, aroylamino, aralkanoylamino, thiol, alkylthio, arylthio, cycloalkylthio, heterocyclothio, alkylthiono, arylthiono, alkylsulfonyl, arylsulfonyl, aralklsulfonyl, sulfonamido, substituted sulfonamido, nitro, cyano, carboxy, carbamyl, substituted carbamyl, alkoxycarbonyl, epoxy or equivalents.

45. The cross linker of claim 42, wherein the functional group provides a secondary linker function to the microporous membrane.

46. The composite microarray slide of claim 40 wherein, the cross linker comprises:
a polyamine.

47. The composite microarray slide of claim 40 wherein, the cross linker comprises:
Epikure 3125, Epikure 3115, Epikure W50, or tetraethylenepentamine.

48. The composite microarray slide of claim 1 wherein, the non-porous substrate comprises:
glass, Mylar, ceramic, acrylic, polypropylene, polycarbonate, polysulfone, polyamide or polyaramid.

49. The composite microarray slide of claim 1 wherein the microporous membrane is selected from the group comprising:
nylon 66, nylon 46, nylon 6, nylon 6-12, nylon polymer blends, polysulfone, polyethersulfone, nitrocellulose, polyvinylidenediflouride (PVDF) or equivalents.

50. A method of fabricating composite microarray slides useful for carrying a microarray of biological polymers comprising the acts of:
providing a non-porous substrate;
providing a microporous membrane formed by a phase inversion process;
providing a surface treatment, wherein the surface treatment comprises organosilanes;
applying the surface treatment to the non-porous substrate; and
operatively associating the non-porous substrate having the surface treatment applied thereto with the microporous membrane for forming an attachment layer therebetween such that the non-porous substrate is sufficiently bonded to the microporous membrane to withstand challenging environments encountered in microarray applications.

51. The method of claim 50 wherein the attachment layer covalently bonds the non-porous substrate and the microporous membrane.

52. The method of claim 50 wherein the microporous membrane substantially covers the surface of the non-porous substrate.

53. The method of claim 50 wherein applying the surface treatment produces an attachment layer having minimal thickness.

54. The surface treatment of claim 53, wherein the attachment layer is between about 0.1 to about 12 microns thick.

55. The surface treatment of claim 53, wherein the attachment layer is between about 2 to about 5 microns thick.

56. The surface treatment of claim 53, wherein the attachment layer is about 3 microns thick.

57. The method of claim 50, wherein the operatively associated of the non-porous substrate to the microporous membrane forms a uniform attachment layer.

58. The method of claim 50 wherein the attachment layer is applied to the non-porous substrate producing a minimal finite thickness or mass which adds uniformity to the overall thickness of the composite microarray slide.

59. The method of claim 50 wherein applying the attachment layer at least substantially eliminates nonuniformity of the overall thickness of the substrate/membrane combination structure.

60. The method of claim 50 further comprising:
providing a microporous membrane containing a sufficient amount of pigments.

61. The microporous membrane of claim 60 wherein the pigments comprise:
carbon-black.

62. The composite microarray slide produced by the method of claim 60 wherein when compared to a microarray slide with a microporous membrane that has substantially no pigments, substantially reduced fluorescence is observed.

63. The composite microarray slide produced by the method of claim 60 wherein when compared to a microarray slide with a microporous membrane that has substantially no pigments, substantially reduced reflectance is observed.

64. The method of claim 50, wherein the microporous membrane, prior to being operatively associated with the non-porous substrate, is wet-as-cast.

65. The method of claim 50, wherein the microporous membrane, prior to being operatively associated with the non-porous substrate, is substantially dry.

66. The microporous membrane of claim 50 wherein the microporous membrane is asymmetric.

67. The microporous membrane of claim 50 wherein the microporous membrane is symmetric.

68. The composite microarray slide of claim 50 wherein the presence of the attachment layer results in minimal interference in the binding of the biological polymer.

69. The method of claim 50 wherein the presence of the attachment layer results in minimal interference the detection of the biological polymers.

70. The method of claim 50 wherein, when subjected to an organic solvent system for greater than about. 6 hours, the microporous membrane does not delaminate significantly from the non-porous substrate.

71. The method of claim 50 wherein when subjected to 4×SSC at about 60° C. for greater than about 10 hours, the microporous membrane does not delaminate significantly from the non-porous substrate.

72. The method of claim 50 wherein when subjected to 4×SSC at about 60° C. for about 2 weeks, the microporous membrane does not delaminate significantly from the non-porous substrate.

73. The method of claim 50 wherein the organosilane is selected from the group comprising:
3-aminopropyltriethoxysilane, 3-aminopropyldimethyethoxysilane, 3-glycidopropyltrimethoxysilane or equivalents.

74. The method of claim 51 wherein, the non-porous substrate is selected from the group comprising:
glass, Mylar, ceramic, acrylic, polypropylene, polycarbonate, polysulfone, polyamide and polyaramid.

75. The method of claim 50 wherein, the non-porous substrate comprises:
glass.

76. The method of claim 50 wherein, the non-porous substrate comprises:
a polyester.

77. The method of claim 50 wherein, the non-porous substrate comprises:
Mylar.
